(19) United States
(12) Patent Application Publication
(10) Pub. No.: US 2026/0201319 A1
(43) Pub. Date: Jul. 16, 2026

(54) ALTERNATIVE STRATEGIES FOR METHANOBACTIN PRODUCTION

(71) Applicants: The Regents of the University of Michigan, Ann Arbor, MI (US); Iowa State University Research Foundation Inc., Ames, IA (US)

(72) Inventors: Jeremy D. SEMRAU, Ann Arbor, MI (US); Alan DISPIRITO, Ames, IA (US); Peng PENG, Ann Arbor, MI (US)

(21) Appl. No.: 19/165,436

(22) PCT Filed: Mar. 15, 2024
(86) PCT No.: PCT/US2024/020119

§ 371 Date: Sep. 15, 2025

Related U.S. Application Data
(60) Provisional application No. 63/452,572, filed on Mar. 16, 2023.

(51) Int. Cl. C12N 1/20; C07K 14/195; C12N 1/32; C12N 9/02; C12N 15/52; C12P 21/00; C12R 1/01

(52) U.S. Cl. CPC C12N 1/20; C07K 14/195; C12N 1/32; C12N 9/0073; C12N 15/52; C12P 21/00; C12Y 114/13025; C12R 2001/01

(57) ABSTRACT

The present disclosure relates to systems and methods for the production of methanobactins. In particular, the disclosure provides modified bacteria and methods of use thereof for production of methanobactin.

### PRIORITY STATEMENT

This application claims priority to U.S. Provisional Application No. 63/452,572, filed Mar. 16, 2023, the entire contents of which are incorporated herein by reference for all purposes.

### STATEMENT REGARDING GOVERNMENT INTEREST

This invention was made with government support under DE-SC0020174 awarded by the U.S. Department of Energy and 1912482 awarded by the U.S. National Science Foundation. The government has certain rights in the invention.

### SEQUENCE LISTING

The text of the computer readable sequence listing filed herewith, titled “UM_41637_601_SequenceListing”, created Mar. 15, 2024, having a file size of 62,754 bytes, is hereby incorporated by reference in its entirety.

### FIELD

The present disclosure relates to systems and methods for the production of methanobactin. In particular, the disclosure provides modified bacteria and methods of use thereof for production of methanobactin.

### BACKGROUND

Methanobactins are low molecular mass copper-binding molecules produced by many methanotrophic bacteria. Methanobactins are used for the treatment of copper-related human diseases, including Wilson disease. However, production of methanobactin is limited by the low natural production rate of methanobactin by methanotrophs, and by the fact that methane, an explosive agent in when combined with air, is used as the growth substrate. Accordingly, what is needed are modified organisms and methods for improved production of methanobactin.

### SUMMARY

In some aspects, provided herein are mutant organisms and methods of using the same for production of methanobactin. In some aspects, provided herein are mutant organisms that produce methanobactin when cultured in the presence of methanol. Accordingly, the mutant organisms provided herein enable large-scale production of methanobactin using methanol, a significantly safer agent than methane, to promote methanobactin production.

In some embodiments, provided herein are mutant organisms having attenuated expression of a gene encoding methane monooxygenase component D (MMOD). The mutant organisms produce methanobactin when cultured in the presence of methanol. In some embodiments, the mutant organism is a methylotroph. In some embodiments, the mutant organism is a methylotrophic bacterium.

In some embodiments, the mutant organism is a methylotrophic bacterium of a class selected from Gammaproteobacteria and Alphaproteobacteria.

In some embodiments, the mutant organism is a member of the Methylocystaceae family, the Beijernickiaceae family, or the Methylobacteriaceae family of bacteria. In some embodiments, the mutant organism is a member of the Methylocystaceae family of bacteria and belongs to a genus selected from Albibacter, Hansschlegelia, Methylocystis, Methylopila, Methylosinus, Pleomorphomonas, and Terasakiella. In some embodiments, the mutant organism is a strain of Methylosinus trichosporium. In some embodiments, the mutant organism is a member of the Methylobacteriaceae family of bacteria and belongs to the genus Methylorubrum. In some embodiments, the mutant organism is a strain Methylorubrum extorquens.

In some embodiments, the mutant organism is a member of the Methylococcaceae family of bacteria. In some embodiments, the mutant organism is a strain of Methylococcus capsulatus (Bath).

In some embodiments, the gene encoding MMOD comprises mmoD. In some embodiments, attenuated expression of the gene encoding MMOD comprises a deletion of mmoD.

In some embodiments, the mutant organism produces at least 50% more methanobactin compared to a wildtype form of the organism when cultured in the presence of methanol.

In some embodiments, provided herein are mutant organisms having attenuated expression of one or more native genes in a methanobactin gene cluster. In some embodiments, the mutant organism having attenuated expression of one or more native genes in a methanobactin gene cluster expresses one or more heterologous genes in a methanobactin gene cluster from a different species of organism. The mutant organism produces methanobactin when cultured in the presence of methanol. In some embodiments, the mutant organism has attenuated expression of three or more native genes in the methanobactin gene cluster and expresses three or more heterologous genes in the methanobactin gene cluster from the different species of organism. In some embodiments, the mutant organism has attenuated expression of each native gene in the methanobactin gene cluster and expresses each heterologous gene in the methanobactin gene cluster from the different species of organism.

In some embodiments, the mutant organism is a Methylosinus species or a Methylorubrum species. In some embodiments, the mutant organism is a strain of Methylosinus trichosporium or Methylorubrum extorquens.

In some embodiments, the one or more native genes are selected from mbnA, mbnB, mbnC, mbnM, mbnN, and combinations thereof. In some embodiments, the one or more native genes are selected from mbnA, mbnB, mbnC, mbnM, mbnN, mbnP, mbnH, and combinations thereof. For example, in some embodiments the mutant organism has attenuated expression of native mbnA, mbnB, mbnC, mbnM, and mbnN.

In some embodiments, the different species of organism is a Methylocystis species.

In some embodiments, the different species of organism is Methylocystis sp. strain SB2.

In some embodiments, the one or more heterologous genes are selected from mbnA, mbnB, mbnC, mbnM, mbnF, mbnS, mbnP, and mbnH.

In some embodiments, the mutant organism is a strain of Methylosinus trichosporium having attenuated expression of native mbnA, mbnB, mbnC, mbnM, and mbnN, and the one or more heterologous genes comprise mbnA, mbnB, mbnC, mbnM, mbnF, mbnS, mbnP, and mbnH from Methylocystis sp. strain SB2.

In some embodiments, the mutant organism further has attenuated expression of a native gene encoding methane monooxygenase component D (MMOD). In some embodiments, attenuated expression of a native gene encoding MMOD comprises a deletion of mmoD. In some embodiments, the mutant organism produces at least 50% more methanobactin compared to a wildtype form of the organism when cultured in the presence of methanol.

In some aspects, provided herein are uses of the mutant organisms provided herein. In some embodiments, provided herein is a use of a mutant organism described herein in a method of producing methanobactin. In some embodiments, the method of producing methanobactin comprises culturing the mutant organism in the presence of methanol.

In some aspects, provided herein are methods of producing methanobactin. In some embodiments, methods of producing methanobactin comprise culturing a mutant organism described herein in the presence of methanol, such that methanobactin is produced. In some embodiments, methods of producing methanobactin comprise culturing the mutant organism in the presence of methanol, such that methanobactin is produced. In some embodiments, the mutant organism produces 50% more methanobactin compared to a wildtype form of the organism when cultured in the presence of methanol and/or in the presence of methanol and copper.

### DESCRIPTION OF THE DRAWINGS

FIG. 1 is a schematic showing the mmo gene cluster of M. trichosporium OB3b. The protein encoded by each gene and the σ⁵⁴ promoter (Pσ⁵⁴) region are indicated in the gene cluster.

FIG. 2 shows a schematic representation of the construction of M. trichosporium OB3b ΔmmoD mutant.

FIG. 3 shows verification of the targeted mmoD gene deletion in M. trichosporium OB3b ΔmmoD mutant by PCR with genomic DNAs extracted from M. trichosporium OB3b ΔmmoD mutant and wild type (WT). The deletion region and PCR region for mutant construction and verification were indicated in the mmmo gene cluster.

FIG. 4 is a bar graph showing RT-qPCR analysis of the relative expression of mbnA in M. trichosporium OB3b wild type (WT) (left panel) and ΔmmoD mutant (right panel) grown with and without 1 μM copper. Error bars indicate standard deviations from triplicate biological cultures.

FIGS. 5A-5B are graphs showing UV-visible absorption spectra of the MB produced from M. trichosporium OB3b wild type (WT, FIG. 5A) and ΔmmoD mutant (FIG. 5B) grown with methane and methanol (without copper).

FIGS. 6A-6B show primary structures of methanobactin from (FIG. 6A) M. trichosporium OB3b and (FIG. 6B) Methylocystis sp. strain SB2.

FIGS. 7A-7B show the mbn gene cluster of M. trichosporium OB3b (FIG. 7A) and Methylocystis sp. strain SB2 (FIG. 7B). Note that function of some gene products have yet to be experimentally determined and are indicated by a question mark.

FIG. 8. UV-visible absorption spectra of the supernatant of ΔmbnAN-mbnAS+pTJS-mbnPH and ΔmmoD ΔmbnAN-mbnAS+pTJS-mbnPH grown with methane or methanol.

FIG. 9A shows UV-visible absorption spectra of 75 μM MB-SB2 from M. trichosporium OB3b ΔmmoD ΔmbnAN-mbnAS+pTJS-mbnPH (MB-AS+PH; red trace), 75 μM MB-SB2 (blue trace) and 100 μM MB-OB3b. FIG. 9B shows UV-visible absorption spectra of 75 μM M. trichosporium OB3b ΔmmoD ΔmbnAN-mbnAS+pTJS-mbnPH as isolated and following 24 0.05 molar additions of CuCl₂.

FIG. 10A shows UV-visible absorption spectra of 75 μM MB-SB2 from M. trichosporium OB3b ΔmmoD ΔmbnAN-mbnAS+pTJS-mbnPH in 100 μM acetic acid and scanned every 20 minutes for 10 hours at 25° C. FIG. 10B shows UV-visible absorption spectra of 75 μM MB-SB2 from M. trichosporium OB3b ΔmmoD ΔmbnAN-mbnAS+pTJS-mbnPH following exposure to 100 μM acidic acid for 10 hours at 25° C.

FIG. 11 shows a mass spectrum of MB as isolated from M. trichosporium OB3b ΔmmoD ΔmbnAN-mbnAS+pTJS-mbnPH and following the addition of 0.6 mole CuCl₂ per μmole of MB from M. trichosporium OB3b ΔmmoD ΔmbnAN-mbnAS+pTJS-mbnPH.

FIG. 12A shows the mbn gene cluster of Methylocystis sp. strain SB2. FIG. 12B shows analysis of the promoter location and transcription rate. The promoter prediction and calculation were conducted using Promoter Calculator.

FIG. 13A shows the mbn gene cluster of Methylocystis sp. strain SB2. FIG. 13B shows the ribosome binding site (RBS) and translation rate. The RBS prediction and calculation were conducted using RBS Calculator.

FIG. 14A is a schematic representation of the construction of M. trichosporium OB3b ΔmbnAN-mbnAS and ΔmmoD ΔmbnAN-mbnAS. FIG. 14B is a schematic representation of the construction of M. trichosporium OB3b ΔmbnAN-mbnAS+pTJS-mbnPH and ΔmmoD ΔmbnAN-mbnAS+pTJS-mbnPH.

FIG. 15 shows results of PCR of mbnAS of Methylocystis sp. SB2 (mbnAS-SB2) in ΔmbnAN-mbnAS and ΔmmoD ΔmbnAN-mbnAS (left). PCR of mbnA of M. trichosporium OB3b (mbnA-OB3b) in ΔmmoD ΔmbnAN-mbnAS and wild type M. trichosporium OB3b (right).

FIG. 16 shows transcriptional analysis (RT-PCR) of the genes of mbn gene cluster of Methylocystis sp. SB2 in ΔmbnAN-mbnAS and ΔmmoD ΔmbnAN-mbnAS+pTJS-mbnPH grown with methane (no Cu added). The bottom blots show a DNA contamination check in the RNA extracted from ΔmbnAN-mbnAS and ΔmmoD ΔmbnAN-mbnAS+pTJS-mbnPH. cDNA and RNA were used as template for PCR of the pmoA of M. trichosporium OB3b.

FIG. 17 shows UV-vis measurement of the supernatant of the culture of ΔmbnAN-mbnAS and ΔmmoD ΔmbnAN-mbnAS grown with methane (without copper).

FIG. 18 shows PCR of mbnP of Methylocystis sp. SB2 in ΔmbnAN-mbnAS+pTJS-mbnPH and ΔmmoD ΔmbnAN-mbnAS+pTJS-mbnPH.

FIG. 19 shows transcriptional analysis (RT-PCR) of mbn genes of Methylocystis sp. SB2 in ΔmbnAN-mbnAS+pTJS-mbnPH and ΔmmoD ΔmbnAN-mbnAS+pTJS-mbnPH grown with methane (no Cu added).

FIG. 20A, FIG. 20D, and FIG. 20E show three different constructs wherein methanobactin genes from Methylocystis sp. strain SB2 were knocked into the chromosome of mutant of Methylosinus trichosporium OB3b with native methanobactin genes and mmoD deleted. FIG. 20B shows UV-visible absorption spectra of the supernatant of the mutant construct. FIG. 20C shows colony PCR from a sucrose plate confirming deletion of native genes and successful insertion of heterologous genes.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “attenuated” refers to being reduced in amount, degree, intensity, or strength. Attenuated gene expression may refer to a significantly reduced amount of a gene, and/or rate of transcription of the gene in question. Attenuated gene expression may also or alternatively refer to a reduced amount of translation, folding, or assembly of the protein encoded by the gene. As nonlimiting examples, an attenuated gene may be a mutated or disrupted gene (e.g., a gene disrupted by a mutation, such as a partial or total deletion, truncation, substitution, frameshifting, or insertional mutation) that does not encode a complete functional open reading frame or that has decreased expression due to alteration or disruption of gene regulatory sequences. An attenuated gene may also be a gene targeted by a construct that reduces expression of the gene, such as, for example, an antisense RNA, microRNA, RNAi molecule, or ribozyme. Attenuated gene expression can be gene expression that is eliminated, for example, reduced to an amount that is insignificant or undetectable. Attenuated gene expression includes a deletion of a gene. Attenuated gene expression can also be gene expression that results in an RNA or protein that is not fully functional or nonfunctional, for example, attenuated gene expression can be gene expression that results in a truncated RNA and/or polypeptide.

The term “heterologous gene expression” or “heterologous expression” refer to expression of one or more genes in an organism that does not natively express the one or more genes (i.e. the one or more genes are not expressed in the wildtype organism).

The term “methanobactin” or “MB” are used interchangeably herein in the broadest sense and refer to a copper-binding peptide produced by methanotrophic organisms. Multiple types of methanobactins (“mbs”) have been identified, including group I and group II methanobactins. The term “methanobactin” is inclusive of all methanobactin types.

The term “methanobactin gene cluster” or “MB gene cluster” as used herein refers to a cluster of genes including mbnA, which encodes a polypeptide precursor to methanobactin, along with at least the genes mbnB and mbnC. In some embodiments, a methanobactin gene cluster includes mbnA, mbnB, and mbnC, along with one or more additional genes. The precise genes in a given methanobactin gene cluster may depend on the species from which the methanobactin gene cluster is determined. In some embodiments, a methanobactin gene cluster includes mbnA, mbnB, mbnC, and mnbnM. In some embodiments, a methanobactin gene cluster includes nmbnA, mbnB, mbnC, mbnM, and mbnN. In some embodiments, a methanobactin gene cluster includes mbnA, mbnB, mbnC, mbnM, mbnF, and mbnS. In some embodiments, a methanobactin gene cluster includes mbnA, mbnB, mbnC, nbnM, mbnF, nbnS, mbnP, and nbnH. In some embodiments, a methanobactin gene cluster includes mbnA, mbnB, mbnC, mbnM, mbnN, mbnP, and mbnH.

As used herein, the term “methylotroph” or “methylotrophic” refers to an organism that uses single carbon compounds (e.g. carbon monoxide, formaldehyde, formamide, formic acid, methanol, methane, methylamine, methyl halide) and/or multi-carbon compounds that contain no carbon-carbon bonds (e.g. dimethyl ether, dimethylamine, dimethyl sulfide, tetramethylammonium, trimethylamine, trimethyl N-oxide, trimethylsulphonium) as their carbon and energy source. As used herein, the term “methanotroph” or “methanotrophic” refers to an organism that metabolizes single carbon compounds as their sole carbon and energy source. Accordingly, methanotrophic organisms require single carbon compounds (e.g. methane, methanol) in order to survive. “Methanotrophs” are considered a subgroup of “methylotrophs”. Accordingly, the term “methylotroph” or “methylotrophic” is also inclusive of “methanotroph” or “methanotrophic”.

As used herein, the term “wild-type” refers to a form found in nature. For example, a wild-type bacterium may be the bacterium as it exists or is found in nature, that is not intentionally modified by human manipulation.

### DETAILED DESCRIPTION

In some aspects, provided herein are mutant organisms with significantly enhanced methanobactin production. The mutant organisms provided herein are able to produce large amounts of methanobactin when methanol is used as the growth substrate. Substitution of a liquid substrate (methanol) for a gaseous and explosive substrate (methane) can greatly facilitate methanobactin production at the commercial scale, such as for production of methanobactin as a therapeutic agent to treat copper-related disorders.

Various types of methanobactins have been identified, including group I methanobactins and group II methanobactins. Reference to enhanced methanobactin production is inclusive of enhanced production of any methanobactin, including a group I methanobactin, a group II methanobactin, or both. An overview of different examples of methanobactins, along with a discussion of differences between methanobactins, is provided in Semrau et al., FEMS Microbiology Letters, Volume 367, Issue 5, March 2020, the entire contents of which are incorporated herein by reference for all purposes. In some embodiments, provided herein are mutant organisms with significantly enhanced group I methanobactin production. In some embodiments, provided herein are mutant organisms with significantly enhanced group II methanobactin production.

Methanobactins are peptides characterized by the presence of one oxazolone ring and a second oxazolone, imidazolone or pyrazinedione ring. The two rings are separated by 2-5 amino acid residues. Group I methanobactins are represented by methanobactin from Methylosinus trichosporium OB3b. Based on sequence similarity and alignments, the putative methanobactins from Methylosinus sp. strain LW3 (mb-LW3), Methylosinus sp. strain LW4 (mb-LW4), Methylosinus sp. strain PW1 (mb-PW1), Methylocystis strain LW5 (mb-LW5) and one of the two mbs from Methylocystis parvus OBBP (mb-OBBP(2)) also fall within this group. In this group the rings are separated by 4 or 5 amino acids and the mb contains 2 or more Cys not involved in ring formation. Group I methanobactins are discussed in in U.S. 20120369809A1, the entire contents of which are incorporated herein by reference for all purposes.

Group II methanobactins are represented by the structurally characterized mbs from Methylocystis strains SB2, rosea and M, and Methylocystis hirsuta CSC1. This mb group lack the Cys in the core peptide, are smaller and likely less rigid, due to the absence of the disulfide bond found in mb-OB3b. In this group the rings are separated by two amino acids. Group II methanobactins are discussed in in U.S. 20120369809A1, the entire contents of which are incorporated herein by reference for all purposes.

In some embodiments, provided herein are mutant organisms that naturally produce methanobactins (e.g. the wildtype organism produces methanobactin), but have been modified to enhance the production of methanobactins (e.g. to produce more methanobactin). In some embodiments, provided herein are mutant organisms that produce group I methanobactins in their wildtype form, but have been modified to produce group II methanobactins.

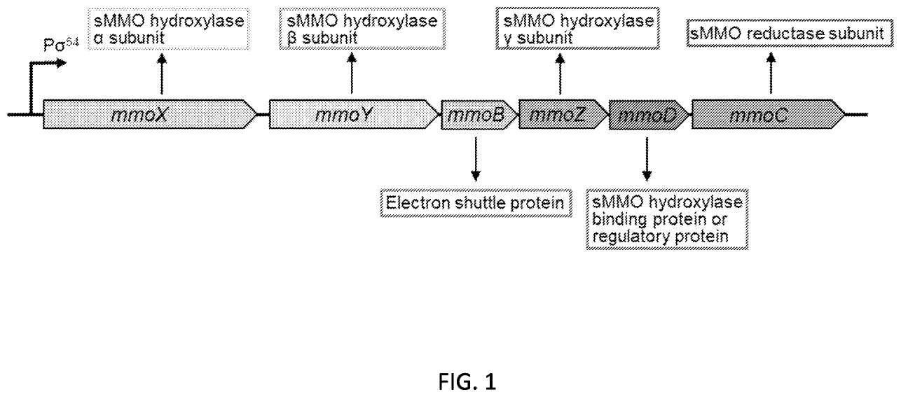

The first step for methane metabolism is the oxidation of methane to methanol. The responsible enzyme for this step/reaction is methane monooxygenase (MMO). There are two types of MMOs: a membrane-bound or particulate MMO (pMMO) and a cytoplasmic or soluble MMO (sMMO). sMMO has three components encoded by the mmo gene cluster: the hydroxylase (MMOH) encoded by mmoXYZ, a reductase encoded by mmoC, and an electron shuttle protein encoded by mmoB (10, 17) (FIG. 1). In addition to these polypeptides, MmoD is also encoded in the mmo gene cluster (FIG. 1). MmoD is not involved in the catalysis of sMMO (10, 17, 22) and is involved in regulating gene expression.

In some aspects, provided herein are mutant organisms having attenuated expression of a gene encoding methane monooxygenase component D (MMOD). The precise sequence of MMOD may vary depending on the species of mutant organism used. In some embodiments, MMOD is defined by an amino acid sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to MRSGGRVARTTKKSGRTKMDQQTAQPEVRQTLIHADERYQAYTMDLEYMLRWEILRD GEFVQEGCSLSQESAREAVAHVLSHFRRQDATSQNDGGKSEAIRALLREIGTPEPLKDEN GAAKPAHI (SEQ ID NO: 1). SEQ ID NO: 1 corresponds to the sequence of MMOD from M. trichosporium OB3b encoded by the mmoD gene. In some embodiments, MMOD is a protein having at least 80%, at least 85%, at least 90%, at least 91%, at least 92%, at last 93%, at least 94%, at least 95%, at least 96%, at least 97%, at least 98%, at least 99%, or 100% identity to SEQ ID NO: 1.

In some embodiments, provided herein is a mutant organism having attenuated expression of a gene encoding methane monooxygenase component D (MMOD), wherein the mutant organism produces methanobactin when cultured in the presence of methanol. In some embodiments, the mutant organism is a methylotroph. In some embodiments, the mutant organism is a methylotrophic bacterium.

In some embodiments, attenuated expression of the gene encoding MMOD comprises one or more mutations in the gene. In some embodiments, attenuated expression of the gene encoding MMOD comprises a deletion of the gene. Any suitable method of attenuating expression of a gene encoding MMOD can be used, including generating gene knock-out technologies (e.g. completely eliminating the gene), and gene silencing techniques. Gene silencing refers to reducing expression of a gene without completely eliminating the gene, as in a knock-out. Suitable methods for attenuating expression include, for example, RNA interference (RNAi), CRISPR, siRNA, antisense oligonucleotides, ribozymes, homologous recombination, and use of site-specific nucleases (e.g. Zing-fingers, TALENS).

In some embodiments, the gene encoding MMOD is the mmoD gene. The mmoD gene is also referred to as orfY. In some embodiments, attenuated expression of the gene encoding MMOD comprises a deletion of the mmoD gene. In some embodiments, the DNA sequence of the mmoD gene is ATGCGTTCGGGCGGCCGCGTCGCCCGAACGACAAAGAAAAGCGGGAGGACGAAAA TGGACCAACAGACGGCGCAGCCGGAAGTCCGGCAAACCTTGATTCACGCCGACGAG CGCTATCAAGCCTATACGATGGACCTCGAATATATGTTGCGATGGGAGATTCTGCGC GACGGCGAGTTCGTGCAGGAGGGCTGCTCTCTGTCGCAGGAGTCGGCGCGCGAGGC GGTCGCTCATGTGCTGAGCCACTTTCGCCGACAGGATGCTACGTCGCAGAACGACG GCGGGAAAAGCGAAGCGATCCGCGCGCTGCTGCGCGAGATCGGAACGCCCGAGCCC CTGAAGGACGAGAACGGCGCGGCG AAGCCGGCGCATATTTAG (SEQ ID NO: 2). In some embodiments, the mmoD gene has at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 2.

In some embodiments, a deletion of the mmoD gene comprises a deletion of a DNA sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 2.

In some embodiments, the mutant organism is a methylotrophic bacterium of a class selected from Gammaproteobacteria and Alphaproteobacteria. In some embodiments, the mutant organism is a methylotrophic bacterium of the Alphaproteobacteria class. The Alphaproteobacteria class includes the Methylocystaceae family, the Beijernickiaceae family, and the Methylobacteriaceae family. In some embodiments, the mutant organism is a member of the Methylocystaceae family of bacteria. In some embodiments, the mutant organism is a member of the Beijernickiaceae family of bacteria. The Methyocystaceae and Beijernickiaceae families are bacteria capable of obtaining carbon and energy from methane. In particular, Methylocystaceae and Beijernickiaceae comprise type II methanotrophs, which utilize the serine pathway of carbon assimilation and characteristically have a system of internal membranes within which methane oxidation occurs.

In some embodiments, the mutant organism is a member of the Beijernickiaceae family of bacteria and belongs to a genus selected from Beijerinckia, Methylocapsa, Methylocella, Methyloferula, Methylorosula, Methylovirgula, and Pseudochelatococcus. In some embodiments, the mutant organism is a strain of Methyloferula stellata. In some embodiments, the mutant organism is a strain of Methylocella silvestris.

In some embodiments, the mutant organism is a member of the Methylocystaceae family of bacteria and belongs to a genus selected from Albibacter, Hansschlegelia, Methylocystis, Methylopila, Methylosinus, Pleomorphomonas, and Terasakiella. In some embodiments, the mutant organism is a Methylosinus species. For example, in some embodiments, the mutant organism is a strain of Methylosinus trichosporium. As another example, in some embodiments, the mutant organism is Methylosinus sp. LW4, Methylosinus sp. LW3, or Methylosinus sp. PW1. In some embodiments, the mutant organism is a Methylocystis species. For example, in some embodiments, the mutant organism is Methylocystis sp. LW5, Methylocystis parvus, Methylocystis SB2, Methylocystis rosea, or Methylocystis SC2.

In some embodiments, the mutant organism is a member of the Methylobacteriaceae family of bacteria. In some embodiments, the mutant organism is a Methylorubrum species. In some embodiments, the mutant organism is Methylorubrum extorquens, Methylorubrum populi, Methylorubrum aminovorans, Methylorubrum pseudosasae, Methylorubrum salsuginis, Methylorubrum suomniense, or Methylorubrum thiocyanatum.

In some embodiments, the mutant organism is a member of the Gammaproteobacteria family. The gammaproteobacterial family includes Type I methanotrophs and Type X methanotrophs, which use the RuMP pathway to assimilate carbon. Exemplary Gammaproteobacteria families include Methylicorpusculum, Methylobacter, Methylocaldum, Methylococcus, Methylocucumis, Methylogaea, Methyloglobulus, Methylolobus, Methylomnagnum, Methylomarinum, Methylomicrobium, Methylononas, Methyloparacoccus, Methyloprofundus, Methylosarcina, Methylosoma, Methylosphaera, Candidatus Methylospira, Methyloterricola, Methylotetracoccus, Methylothermus, Methylovarius, and Methylovulun.

In some embodiments, the mutant organism is a Gammaproteobacteria selected from the Methylococcaceae family and Methylothermaceae family. In some embodiments, the mutant organism is a strain of Methylococcus capsulatus (bath). In some embodiments, the mutant organism is a strain of Methylomagnun ishizawai, Methylotuvimicrobium buryatense, Methylovulum miyakonense, Methylomonas methicanica, Methylomonas sp LW13, Methylononas sp. MK1, or Methylomonas sp. 1 lb.

In some embodiments, the mutant organism produces at least 50% more methanobactin compared to a wildtype form of the organism when cultured in the presence of methanol. For example, in some embodiments the mutant organism produces at least 50% more, at least 55% more, at least 60% more, at least 65% more, at least 70% more, at least 75% more, at least 80% more, at least 85% more, at least 90% more, at least 95% more, at least 100% more, or more than 100% more (e.g. more than double the amount) methanobactin compared to a wildtype form of the organism when cultured in the presence of methanol. In some embodiments, the mutant organism produces more than 3 times, more than 4 times, or more than 5 times the amount of methanobactin compared to a wildtype form of the organism when cultured in the presence of methanol. In some embodiments, the mutant organism is able to produce methanobactin when cultured in the presence of methanol or when cultured in the presence of methane.

In some embodiments, the mutant organism produces at least 50% more methanobactin compared to a wildtype form of the organism when cultured in the presence of methanol, regardless of whether copper is present or absent in the growth conditions. For example, in some embodiments the mutant organism produces at least 50% more, at least 55% more, at least 60% more, at least 65% more, at least 70% more, at least 75% more, at least 80% more, at least 85% more, at least 90% more, at least 95% more, at least 100% more, or more than 100% more (e.g. more than double the amount) methanobactin compared to a wildtype form of the organism when cultured in the presence of methanol, regardless of whether copper is present or absent in the growth conditions. In some embodiments, the mutant organism produces more than 3 times, more than 4 times, or more than 5 times the amount of methanobactin compared to a wildtype form of the organism when cultured in the presence of methanol, regardless of whether copper is present or absent in the growth conditions. In some embodiments it may be advantageous to use copper in the growth conditions to facilitate greater production of methanobactin. In some embodiments, it may be advantageous to not use copper in the growth conditions.

In some embodiments, the mutant organism has attenuated expression of a gene encoding MMOD (e.g. a deletion of mmoD), as described above, along with one or more additional modifications. In some embodiments, the mutant organism has attenuated expression of a gene encoding MMOD (e.g. a deletion of mmoD) as described above, and also heterologously expresses of one or more genes in a methanobactin gene cluster from a different species of organism, as described in more detail below. The terms “heterologous expression”, “heterologously expresses”, and the like are used interchangeably herein and refer to expression of a non-native gene in an organism. The non-native gene may be derived from a different species of organism. In some embodiments, the mutant organism has attenuated expression of a gene encoding MMOD (e.g. a deletion of mmoD) as described above also has attenuated expression of one or more native genes in a methanobactin gene cluster (e.g. deletion of one or more native genes in the methanobactin gene cluster) and the one or more native genes are replaced by heterologous expression of a gene in a methanobactin gene cluster from a different species, as described in more detail below.

The mbnA gene encodes a polypeptide precursor of mature methanobactin. mbnA is part of a methanobactin gene cluster that also includes mbnB and mbnC, which are believed to be involved in synthesis of the C-terminal oxazolone ring of methanobactins. In some aspects, provided herein are mutant organisms having attenuated expression of one or more genes in a methanobactin gene cluster. In some embodiments, attenuated expression of the one or more genes indicates that the one or more genes are deleted in the mutant organism. The mutant organisms having attenuated expression of one or more genes in a methanobactin gene cluster may also have attenuated expression of a gene encoding MMOD, as described above.

Exemplary genes that may be included in a methanobactin gene cluster are mbnA, mbnB, mbnC, mbnM, mbnP, mbnH, mbnI, mbnR, mbnT, mbnF, mbnN, and mbnS. The specific genes in a given methanobactin gene cluster depend on the specific organism (e.g. methanotroph) in which the gene cluster exists. The specific sequence of a given gene in a methanobactin gene cluster (e.g. mbnA, mbnB, mbnC, etc.) may also vary depending on the precise species of mutant organism. Exemplary methanobactin genes and methanobactin gene clusters are described in Semrau et al., FEMS Microbiology Letters, Volume 367, Issue 5, March 2020, the entire contents of which are incorporated herein by reference for all purposes. In some embodiments, the methanobactin gene cluster (MB gene cluster) comprises mbnA, mbnB and mbnC, along with one or more additional genes. In some embodiments, provided herein is a mutant organism having attenuated expression of one or more native genes in a methanobactin gene cluster, wherein the mutant organism expresses one or more heterologous genes in a methanobactin gene cluster from a different species of organism (e.g. the one or more native genes in a methanobactin gene cluster are replaced by one or more heterologous genes in a methanobactin gene cluster from a different species). For example, in some embodiments provided herein is mutant organism “A” having attenuated expression of one or more native genes in “methanobactin gene cluster A”, wherein the mutant organism “A” expresses one or more heterologous genes in “methanobactin gene cluster B” from organism “B”.

In some embodiments, provided herein is a mutant organism having attenuated expression of two or more native genes in a methanobactin gene cluster. In some embodiments, the mutant organism expresses two or more heterologous genes in a methanobactin gene cluster from a different organism. In some embodiments, provided herein is a mutant organism having attenuated expression of three or more native genes in a methanobactin gene cluster. In some embodiments, the mutant organism expresses three or more heterologous genes in a methanobactin gene cluster from a different species of organism. In some embodiments, provided herein is a mutant organism having attenuated expression of four or more native genes in a methanobactin gene cluster. In some embodiments, the mutant organism expresses four or more heterologous genes in a methanobactin gene cluster from a different species of organism. In some embodiments, provided herein is a mutant organism having attenuated expression of five or more native genes in a methanobactin gene cluster. In some embodiments, the mutant organism expresses five or more heterologous genes in a methanobactin gene cluster from a different species of organism. In some embodiments, provided herein is a mutant organism having attenuated expression of each native genes in a methanobactin gene cluster. In some embodiments, the mutant organism expresses each heterologous gene in a methanobactin gene cluster from a different species of organism.

In some embodiments, the methanobactin gene cluster comprises native mbnA, mbnB, mbnC, mnbnM, and mbnN. In some embodiments, the methanobactin gene cluster comprises native mbnA, mbnB, mbnC, mbnM, mbnN, mbnP, and mbnH. The sequences of mbnA, mbnB, mbnC, mbnM, mbnN, mbnP, and mnbnH in M. trichosporium OB3b are provided below:

M. trichosporium OB3b
mbnA
(SEQ ID NO: 3)
ATGACTGTCAAGATTGCTCAGAAGAAAGTCCTTCCGGTCATCGGTCGCGC
GGCTGCGCTTTGCGGTTCGTGCTATCCGTGCAGCTGCATG
mbnB
(SEQ ID NO: 4)
GTGCTCGGGGGCGGCACTCGCCTCTTCGGGGCGATTCTGGCCGTATTCCA
CGAGGCGAAGATGCAGATCGGCTTCAACTTCACGCTGACCGGCACGCTCG
ACATGGTCCAGCAGATGATCAAAGAGCGGAAAATCGACTATGTCGAAATG
TTGATCGACAATTTCGTTCATCTCCCGCCCGAGCAGATCGCGGATTCATT
CGATTGTCCCGTTGCATTTCATATCATGCTTTCGAAATACCTCGAAAGAG
ATCGGGAGGAATTGGAGAAGCTCGGGAAGCGGCTGCGCAGATTCATCGAC
GTGATGCGGCCCGTCTATGTCTCTGATCATATTTTATACTTCACACACAA
CGGCAGAAGCCTTTTCCATCTCGGCGAAATCGATTATTGAGAATACGACC
ACGTGCGGAGCAAGGTCGAGCAATGGCAGGACATGCTCGGCACGCGCCTC
TATCTTGAGAATTATCCGTCGATCATGGATGGCGCATGGGACGCGCCGTC
CTTCTACGAGCGGCTGAGCCGGGAGACCGGGGTCGGCGTTCTCTTCGACG
CGTCGAACGCGATCTGCGCTCAGAACAACACCGGCGCGCCGGTCGAGCTG
TGGAAGAAAATCATCGAGACCACGCGCCATTTCCACGTTGCAGGATATGG
CACGGCTTTCATCGAGCCGCGCGTCAAGGCCGACACGCACGACCGGGAGA
TGGCGGAGGACACGCTCGATTTCCTGAGCCGGATGCGCACGTCTTTCGAC
AAGCCCGGCGCGACGATCACTTATGAACGGGATTTCGACATTGACTATGA
GTCGATCTCGGTCGACCTGAAAAGACTGCGCGATATTTTCCCCTGCGTCG
AGGAAGAGCGTCATGAGCCTGTTGCCCACTGCGCCGGTTAG
mbnC
(SEQ ID NO: 5)
ATGAGCCTGTTGCCCACTGCGCCGGTTAGAATCGACGCCGATCTCTACGA
TGATCTCGCAAATCCTGCCCGCCAGTCTCTCTATCCCAGGGATTCGCGCG
GCTTCATTCGCATCGATATCAGCCTTCGGGCCTACTGGCACACATTGTTC
GACACATGCCCACGCCTGCTGGAGCTCTCGGGGCCGAGCGGCGGCGCCAT
CTTCCTGCCCTTCATGGCCTGGGCCCGGGAGAACAACCTCGCTTTCGACT
GGAGCTTTTTCCTCTGGGTCTATGTCTGGCTGCAGCAATCGGAGTTCCGC
GAGCGGCTCGACGAGGATCAGCTGCTGCCCGTCATGACCGCCTCCGCGAC
GCGATGGTTGATGATCGACCGCGACATCGATGCCTGTCAGATCGTGCTGG
GAAGCCGCTCGCTCGCCGGCGCAGCCGTCGTCGGCGCCAAGATCGACAGC
ATCCACTGCCGGCTCGAGCAGGTCCAGCAGGTCGAATTCGAGAAGCCGCT
GCCGCTCCCCGACGGCGAGTTCGGCTATTTCCTGACGCCCGGCTTCGAGA
TCGACCATTTTCCCGGCTGGCGGCCGCTCCCGCGATGA
mbnM
(SEQ ID NO: 6)
ATGACGCGGCCCAAAACGGGCTTCTGGCGCGGCTACTGGCCATTGCTCAT
GGCTCTGGCCTCGGCTCAACTCGCGCAGCAAGCGGACATCGTGATGATGA
GCCGCCTCGGCGGCCCCGCCGCCGGCGCTTATGTGATGATGATGCGCCTA
GCGCTCGTCGAAGTCGTGCTGATGACGGCGATGGGCGCGGTCGCATCGAC
GGTGGTCGCGCAGGCGCAACGAAACGGCGGGGCCGCGCCGGTCATCGGCC
GCATCCTCGGCCTGGCCCTGCTGACGGGACTGCTCTGCGGCGCGCTAGGC
TGGCTCCTTTATCCCACGGCGGCGAGGTGGCTGGCGGGGGAAGGCGACGT
CGCGAGCCTCGTCGGCGAGGGTGTGTTCTGGCACGCGCTGGCGGCGCCCT
TTCGGTTTGTGGTCAACATCGGCGCATTCGTCCTCCACGCCCTCGGTCGT
GGCCCTTCGGTCGTTCGGTGGAAATTGACCGAGGCGGCGGCCAAGATCGC
CGCCAATCTGCTCCTCATGGATGTTTTGGGATGGGGATTTTCGGGCTGCT
TCTTGTCCGGCCTCATCGTCGGCGCGCTGTCGTCGATCTGGTGCCGCCGC
ATGTTGCCGCCGGGCGACGCGAGGCTTTCGCTTCCCGGATGCTCGTGGAC
GCTGCGCTTCCTTCGTCCGATCTATTGGGAGGCGCAGCGGCTCATATCCG
TGCAGCTCGCCATACTCGCCTGCTTCTCCCTGTTCGCGGCGCCGTGGCTC
GCGAATTACAGCGTCTCGCGGATGAATTCCTTCGCGGCCGGCCAGACCTT
GATGCTGATGCTGTTCACGCCCTTCGTCGCGCTGACGCGATTCCTGGCGT
TCCGGCACGCTGCGCTGCGCGACGGCCGGCTCTCGGCGTCGATGTGGAGA
GTGTGGGCGCAGGGCGCGCCTGTCGTGGTCGCCGCCGCCCTCGCCTTGCT
GGAGAGCGACGAGCTGCTCGGTCGCTTATATGGCCAGAGCGGGCATTGGT
GGTCGACGCTGATCCAGGCGCTGGCGATTTCGCTTCCCCTTCGCTTCGCG
GCGAATATCGCCCGAGCCTTGTTCCATTCGCAGGGCTCGTTCGCCATGGT
CGCAGCCGCCGACGGCGCCGCGTTTTGGCTGATCGCGACGCCGCTTGTCG
CCATCGGGCTCTACGTCGACTCCCCGGCCGTCGCTTATTCCTCGCTGATC
CTTCCAGAAGCGGCCTGCGCCGCTTGGATGTGGGGCCGGCTCGGAATTCT
CGCTTTCCGTGAAAAAGGACCGGTCGGCGCCGACGAAGGCGTGAAAGCTT
TGCTCGAATGA
mbnN
(SEQ ID NO: 7)
ATGACGGCGATCCCATGTGAAACGAACGGGCTCACGAGCGAGCCGATCGC
GATGCGAGCCAAATCGAGCCTGGCCGTCCCGATATTCGCCGTTGCGCCGC
GGCCCGACGGCATCTTGCTCGACAGCAACGAATTTCGCTTTCCTCTCCCG
AAGAGGTTTCTGAAACGCGTCGCGGACGAGCTGTGGCCGGTCGATCTGCG
ATGCTATCCTGATTTCGCCGAAGTGGAAAAAATCAAGGTCGCCTTGGCGC
GAGACCTCGGCGTCGAGCCCGACAATCTGTTGCTCGGGGTCGGCTCGACC
GAACTCCTCGATTTGATCAGCCGCGCTTTCGGCGAAAGGGACCACAGAAT
CGTCACGCCGATTCCATCCTTTCCGATGTATGCTCATTACGCCAATCTGA
ACGATCGAAAATTCGTTCCCGTTCCGCTCGACGACGATCTCTCCTTGTCT
TCGAAAGTGGCGGAGGCGCTGCTGCGGGAGCCCGGGAGCAAATCCTTCTA
TGTCTGCAGCCCGAATAATCCCACCGGGCTTTCCATAGAGCGAGAGACGT
TCGACGCTCTCGCTTCCGATCCTCGCGCCATTTTGTGCCTGGACGAGGCA
TACGCCGAATATTCCGGCCGCTCCTTCGTCACGGAGGCGGCGACACGCGA
CAATGTCGTCGTCACGCGCTCCTTTTCGAAGATCGGCCTCGCGGGATGCC
GGTTCGGCTATGCGGTCGCCTCGAAAAATCTCATCCGGGAACTCGCGAAG
GCCGCTCTGCCCTATGCTCTGAACTCCCTCACCTTGCGCATTGCGCAAAT
CTTCATCGAGGAGATGCCGCTGATCAGAGCGGTCGTCGAGGACGTCAAGC
GTGAACGGGCGCGTCTTTCGACCGCGCTGGCGAACCTGCCCGGTTTCCAC
CCCTACCGATCCGACGCCAACTTCCTGCTCTGCGCGGCGCCTGGCGACGC
CGCCGCGCTCGCCCGAAGGTTGATCGAGCTACGAAAAATCTGGGTGCGAC
CCTTTTCGCCCCAATCGCGCTTACGCGACTGTCTGCGCATCGCGGTCGGT
TCGCCGGAGGACAATGACATCCTTCTAGACGCCTTGAGACAGGAATCGAC
GATCCACGCGCCTCGGTCGGAATGA
mbnP
(SEQ ID NO: 8)
ATGTGGGAATTGCATAGAGCGCGGACCGCGACAATTGCCGCCGGAGCGCT
GATTGTCGCCGGCGCGGCGCTGGCCGCGGGCGTGAAGACACAGCCGGTCG
CAGTCCGTTTCGCGCTCGTCGCCGATGGCAAGGAAGTGGGCTGCGGCGCG
CCGCTCGCAAACCTCGGCAGCGGGCGCCTCGCCGGCAAGCTGCACGAAGC
GCGCCTCTACGTCTATGGCTTCGAGCTGGTCGACGCCAAAGGGAAGCACA
CACCCATCGCACTGACGCAGAACGATTGGCAATACGCCGATGTCGCGCTG
CTCGACTTCAAGGACGCGCGCGGAGGCAACGCCGCTTGCACGCCGGGCAA
TCCGGCCAAGAACACGACCGTCGTCGGAGCCGCGCCGCAAGGCGCCTATG
TCGGTCTCGCCTTCTCTGTCGGCGCCCCGGTGGAGAGCCTCGTCGACGGC
AAGCCCGTCTTCGTCAACCATTCCAATGTCGAGGCCGCGCCGCCGCCTCT
CGACATTTCCGGAATGGCGTGGAACTGGCAGGCCGGCCGCCGGTTCGTGA
CGATCGAGGTCATTCCGCCGGCGGCGGTGATCAAGCCCGACGGCTCCAAG
TCGCGGACCTGGATGGTCCATGTCGGCTCGACCGGCTGCAAGGGCAATCC
GGCGACCGGCGAGATCGTCGCCTGTGCGCATGAGAACCGCTTTCCTGTCG
TCTTCGACCGCTTCGATCCGAAGACGCAGCGCGTCGAGCTCGATTTGACG
ACGCTGTTCGAGAGCAGCGACATCAGCGTCGACAAGGGCGGCGCGGTCGG
CTGCATGAGCGCGCTCGATGATCCCGACTGCCCGGCGGTGTTCCGCGCGC
TCGGCCTCAATCTCGCCGACAGCGCGCCCGGCGCCAATGACGCCGGCAAG
CCATCGAGGCCCGGCGTCTCGCCGATATTCTCTGTCGGCGCCGCAGCGTC
GAAAGTCGCGGGCGGCAAGCAATGA
mbnH
(SEQ ID NO: 9)
ATGATGCGCGCGTCGTTTCTCGTACTCGCCGCGCTCGTTCTCGGCGCGCG
CTCTGCGGCCGAGCCGGCCCCCGCCTGGAATTGGGACTTACCGAAATACA
TCCCGCCGCCGCGTGTTCCCGTCGACAATCCGATGTCGGAGGAGAAGTTC
CAGCTCGGGCGGCGCCTCTTCTACGATAAGCGCCTCTCCGGCAATGGAAC
GCTCTCGTGCAGCTCCTGCCATCTGCAGGAGCGCGCCTTTACCGACGGAC
GCACCGTCTCCATCGGCTCGACGGGCGCGAAGACGCCGCGCAATGCGCCG
TCGATCGCCTATTCCGGCTGGCATGGAACGCTCACCTGGGCCAATCCGGC
GCTGGTGACATTGGAGCGGCAGATGCTCAATCCGCTGTTCGGCGCCGATC
CGATCGAGATGGGCGCGAGCGACGCCAATAAGGCGGAGATCGTCGCGCGC
TTTCGCGCCGACGCCGATTATCGCCGATGGTTCGCCGCGGCCTTTCCCGA
AATGAGCGAGCCCATCTCCTTCGCGACGATCATCGCGGCGATCTCGGCGT
TCCAGCGCGGCGTCTATTCCTTCGACAGCCGCTATGATCATTATCTGCAG
GGCGAGGCGCAGCTCACCGAAGCCGAGCAGCGCGGACATGATCTTTATTT
CGGTGAGAAGGCGGAATGTCATCACTGCCATGGCAGCGTCGGCCTCGACG
ATCAATTCGTGCATGCGAGGACGCGCGAGCCGGAGCTGCCGTTCCATAAC
ACCGGGCTCTACGACATTGACGGAAAGGGCGCTTATCCCGCGCCCAATCA
CGGGCTCTTCGACATCACCGGCGATCCGGACGACATGGGCAAGTTCCGCG
CGCCGAGCCTGCGCAACATCGCGCTGACCGCGCCTTACATGCATGACGGC
AGCGTGGCGACGCTGGAAGAGGTGATCGACATTTATTCCGAAGGCGGGCG
TAAGATCGCGAGCGGGCCGCATGCGGGCGACGGCCGCGCCAGTGCGCTGA
AGAGCGGGCTGATCGTGAAGATCGATCTGACGGCGCAGGAGAAGGCTGAT
CTCCTCGCCTTCCTGAAGACGCTGACCGACGAGAGCCTGATCGCCTCGCC
GCGTTTCTCTGATCCCTGGAGAACGCCGACCGCTGCGAAATGA.

In some embodiments, the mutant organism has attenuated expression of three of more of native mbnA, mbnB, mbnC, mbnM, and mbnN. In some embodiments, the mutant organism has attenuated expression of each of native mbnA, mbnB, mbnC, mbnM, and mbnN. In some embodiments, the mutant organism has attenuated expression of each of native mbnA, mbnB, mbnC, nbnM, mbnN, and does not have attenuated expression of native mbnP and mbnH. In some embodiments, the mutant organism has attenuated expression of each of native mbnA, mbnB, mbnC, mbnM, mbnN, and attenuated expression of native mbnP and mbnH. In some embodiments, the mutant organism is a strain of Methylosinus trichosporium and has attenuated expression of each of native mbnA, mbnB, mbnC, mbnM, and mbnN.

In some embodiments, expression of one or more of native mbnA, mbnB, mbnC, mbnM, mbnN, mbnP, and mbnH is attenuated in the mutant organism. In some embodiments, expression of two or more of native mbnA, mbnB, mbnC, mbnM, mbnN, mbnP, and mbnH is attenuated in the mutant organism. In some embodiments, expression of three or more of native mbnA, mbnB, mbnC, mbnM, mbnN, mbnP, and mbnH is attenuated in the mutant organism. In some embodiments, expression of native mbnA, mbnB, mbnC, mbnM, mbnN, mbnP, and mbnH is attenuated in the mutant organism. In some embodiments, expression of each native gene in the methanobactin gene cluster is attenuated. For example, in some embodiments the mutant organism has attenuated expression of each of mbnA, mbnB, mbnC, mbnM, mbnN, mbnP, and mbnH and attenuated expression of any other methanobactin gene present in the native methanobactin gene cluster. In some embodiments, the mutant organism is a strain of Methylosinus trichosporium.

In some embodiments, attenuated expression of one or more native genes (e.g. two or more native genes, three or more native genes, four or more native genes, five or more native genes, etc.) indicates a deletion of the one or more native genes in the mutant organism. The precise sequence of the native genes in a given methanobactin gene cluster may vary depending on the species of mutant organism. In some embodiments, attenuated expression of the one or more native genes in the methanobactin gene cluster indicates a deletion of one or more sequences each having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) with one or more of SEQ ID NO: 3-SEQ ID NO: 9. For example, in some embodiments attenuated expression of mbnA indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 3, attenuated expression of mbnB indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 4, attenuated expression of mbnC indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 5, attenuated expression of mbnM indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 6, attenuated expression of mbnN indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 7, attenuated expression of mbnP indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 8, and attenuated expression of mbnH indicates a deletion of a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 9.

In some embodiments, the mutant organism heterologously expresses one or more of mbnA, mbnB, mbnC, mbnM, mbnN, mbnF, mbnS, mbnP and mbnH from a different species. For example, in some embodiments one or more of mbnA, mbnB, mbnC, nbnM, mbnN, mbnF, mbnS, mbnP and mbnH is heterologously expressed in the mutant organism to “replace” the one or more native genes (e.g. one or more of mbnA, mbnB, mbnC, mbnM, mbnN, mbnF, mbnS, mbnP and mbnH) having attenuated expression in the mutant organism. In some embodiments, the mutant organism heterologously expresses two or more of mbnA, mbnB, mbnC, mbnM, mbnN, mbnF, mbnS, mbnP and mbnH from a different species. In some embodiments, the mutant organism heterologously expresses three or more of mbnA, mbnB, mbnC, mbnM, mbnN, mbnF, mbnS, mbnP and mbnH from a different species. In some embodiments, the mutant organism heterologously expresses four or more of mbnA, mbnB, mbnC, mbnM, mbnN, mbnF, mbnS, mbnP and mbnH from a different species. In some embodiments, the mutant organism heterologously expresses five or more of mbnA, mbnB, mbnC, mbnM, mbnN, mbnF, mbnS, mbnP and mbnH from a different species. In some embodiments, expression of each of native mbnA, mbnB, mbnC, nbnM, and mbnN is attenuated in the mutant organism (e.g. sequences each having at least 80% sequence identity with SEQ ID NO: 3, SEQ ID NO: 4, SEQ ID NO: 5, SEQ ID NO: 6, and SEQ ID NO: 7 are deleted in the mutant organism) and the mutant organism heterologously expresses each of mbnA, mbnB, mbnC, mbnM, mbnN, mbnF, mbnS, mbnP and mbnH from the different species of organism. In some embodiments, expression of each of native mbnA, mbnB, mbnC, mbnM, mbnN, mbnP, and mnbnH is attenuated in the mutant organisms and the mutant organism heterologously expresses each of mbnA, mbnB, mbnC, mbnM, mbnN, mbnF, mbnS, mbnP and mbnH from the different species of organism. In some embodiments, each native gene in the methanobactin gene cluster is attenuated and the mutant organism expresses each heterologous gene in the methanobactin gene cluster from the different species. In some embodiments, the heterologously expressed methanobactin genes are from Methylocystis species strain SB2. In some embodiments, the mutant organism has attenuated expression of one or more native genes in a methanobactin gene cluster, attenuated expression of a native gene encoding MMOD (e.g. a deletion of mmoD), as described above, and heterologously expresses one or more genes from a methanobactin gene cluster from a different species.

The precise sequence of the heterologous genes (e.g. the genes heterologously expressed in the mutant organism) may vary depending on the species of organism from which the heterologous genes were obtained. The sequences of methanobactin genes in Methylocystis species strain SB2 are provided below:

Methylocystis sp. SB2
mbnA
(SEQ ID NO: 10)
ATGACCATCCGTATCGCCAAGCGCATCACCCTCAATGTCATTGGCCGCGC
CAGCGCCCGCTGCGCGTCGACCTGCGCCGCCACCAACGGCTAA
mbnB
(SEQ ID NO: 11)
CGACTTGGCTTCAATTTCACGCTCGGCGACACCTATGACATGGCTCAGAG
GCTGATCGCCGAAGGGCACATCGATTATTGCGAATTTCTTATCGACAATT
TTTTCTGCGTCGATCCGGACGAATTGGCCAAGGCGTTCGATTGTCCAGTC
GGGTTGCACATTATGTATTCGAAGTTTCTCGAAGCCGATCGGCCGACGCT
CGTCGATTTCGCCGCTCGGCTTCGAGATTACATCGACGCGCTGAACCCGA
TTTATGTCTCCGACCATATTCTGCGCTTTTCTCACGAGGGGCGCGCCTTC
TTTCACTTAGGCGAAATCGATTATTCCGCCGAATATGAATCCGTGCGCGA
CAAGGTCGTGCAATGGCAGGAAATCGTCGGACAGCGCATTCATTTTGAGA
ATTACCCGTCCATCATGGACGGCGGGTTGGAAGCGCCCGCTTTTTTCGAA
CGCCTCATGAAGGAAACAGGCGCGGGCGTGCTGTTCGACGCCTCCAACGC
CGTCTGCGCCAATCGCAATTGCGGGACGCCGTTCGAGGCCTGGCGCAACG
TGATCGCGAAAGCCCAGCATTTCCACGTCGCCGGCTATCGGCAGTCCTTG
ATCGAGCCCTTCATTTCGCTCGACACCCATGCCGAGGCGTTGGCGCCGGA
CACGCTCGCCTTCCTCCAGAATTTTCGATCGGTCTTCGACAAGCCGGGCG
CAACGATGACTTATGAGCGCGACGATCAAATCGAGTTCGACGATATCGTC
GTCGATCTGAAGGCGTTGCGCGATCTGTTCGGCCAACCAGAAGAAACGCG
TCATGACCTTGCTCTCACTGCCTAG
mbnC
(SEQ ID NO: 12)
ATGACCTTGCTCTCACTGCCTAGTCGGACTGATCGCGACCTTTACCCTGC
GCTGATGTCGCCGCAGCTGAACGCAAAATATCCTGCGGACCACAAGGCGT
CGTGCGGATCGACTGTGAGCCTGCGCATCTATTGGCACACGCTCTTCGAC
ATTTGCCCAGAACTGCTGGAGCTTTCCGGTCCGGACGGCATGTCGATTTT
CCGGCCTTTCATGGCCTGGGCTGAAGAGAAGAAGCTCGCCTTCGACTGGT
CCTATTATCTTTGGGTCTATGTTTGGCTGCGCCAATCGCCGTTCAAGGAA
CGGCTGTTGGCGGACGACAAATATCTCCGATCGATCATGGCGGCCTCCGC
CGCGCGGTGGGCGATCCTCGATCGCGGTCGAAGCCGCGGGATCGTGATCG
GATGCGCCGATACGGCGGATCTCGTTATCGGCTGGAAATGCCGCAACGTT
CGCCTCGGACGAGAGATCGAGCTTATCGAACCCGAGGAGCCGTTGCCGGC
GCCGCCAGACCTCTTCGGCTACTTTACGCTGCCGAGCTTTGAACTCGACA
TATTTCCGGGGTGGCAGAGCCTCGCGCGATGA
mbnM
(SEQ ID NO: 13)
ATGACAAGCGCGCCGCTGTGGCGCGGATACTGGTTCCTGTTCGCCAGCAC
GTCCGCAGCCGCCTTCATCATGCAGATCGATCTCGCCATGGTCGCGAGAT
TGGGCGGCCGCGCGTCAGGCGCCTATGCGATTCTGATGCGCGTCACGCTG
CTCGATGTCGCGGTCACCATCGCTTCCGCGGCCGTCGCCGCCATCGCGCT
CGGCCATGCGCAAAAGAAGGGCGAGACGGCCGACGCGATCGAAAAGACCT
GCGCGCTCTCCCTCGCGCTTGGCGTCGCGACAGCGATTTTCGGCTTCTTC
GCCTATCCGATCTTCCTTGACGCCCTGATGGGCGACGCCGCCGCCGCTCC
CTATATCGGCGCCCCGATCCTTTGGCTCGTCGCCGGCGCGCCGTTCCGCG
TTCTATCGAACACGCAAGGATTCCTGTTGCACGCGCTTGGACGCGGCGGC
TCGGTTCTCACATGGAAGCTCGCAGAAATTCCAGCGAAAGCCGGCGCCAA
CGTCCTGTTCATGCAAACCTTCGGATTGGGTTTTACCGGCTGTTTTCTCG
CGGGCTGCGTCGTCGCCCTCGCCTCGTCTTTTTGGTTATGGAGTCGGCTG
CACGCGCATGGCGCGCGCACATTGCGCCTTCCCGAACTCGCCTTCGCGCG
CTCCTTTCTGAGCGGAACCTTCTGGGAAGCGCTGCGGGTGCTGTCGCCGC
AGGCGGCGATGCTCTTCTCGCTGACGCTCTTTTCGCTGCCCTGGCATTAC
GCCGCGAACGGCCAACGCCTCGACTCCTACGCGGCCGCGCAGACCTTCAT
GCTGTTCATTCTTGGTCCGCTCATTACGCTGATGCGCTATCTCGCCATTT
ACCTTCCCAAGCAATCTCCCGAGGATTGGACGCCAGCCCTTGCGCCGGTG
ATGCGCGTCGGCGCGCCACTCGCGCTGACGGCCGCCGTCTTGCTGCTATC
GGGGCGCGATTGGATCGGGGACGCTCTCTACCGCCAACACGGACCCTGGT
GGTCGGTTTTCGTCGCCTGCTTGGCGATTTCCATACCTATCCGATTCATC
GGCAGCATCGTCCGAGGACTAACGCTCGCTCGGAGCGCCTTCGCAGAACT
GACCTCCGTCGATGTGCTTGCGCAGTGGCTCATCGCGCCGCTGTTCATTC
TGTTCGGCCTCTCCGTAAACCGTCCGGAGATCGCCTATCAATCGCTGATC
TGGCCTGAAATCGTCGCTGTTTTCTTGCTCTGGCGCGGACTTCGATCCGC
GCCCGAAGAGCCCGTCGGCGTCCCGTGGCCCGCTAAAGGACATGTGCAAT
GA
mbnF
(SEQ ID NO: 14)
ATGAGCGTGGAGCGAGTCCCGGTTCTGATCGTCGGCGCCGGCTATGCCGG
TCTGTCTGCGGCGACGTTGCTTGCATGGCGCGGCGTCCCCTGCCGTCTCG
TCGAGCGACGCGCGTCAACGTCGCGACTGCCGAAAGCGCATGGGATCAAC
CGCCGCAGCATGGAAGTTCTGCGCGTCGTCCCTGGCTTGGAGGACGCGTT
GTTTGCGGCAAGTCGCGCCGGCGCCAACGAATCCACGCTCATCATCGCCG
AGTCGGTCACGAGCCCGCCGATCGAAACGCTCGTCACCAAGATCTCGCTC
GACGCAACGCCGGTGTCTCCCAGCAGAATATGCACCGCAGGCCAGGATCG
AGTCGAACCCGTGCTGCTGCGCTTCGCCCGCGAGAACGGCGCCGACGTTC
GATTTTCGACGACGCTAGAGCGCTTCTCGCAGCGGGACGACGGCGTCGAC
GCGATCCTGCGCGATGAAGCGTCGGGACAAGAGACCACCGTTCTCGCGGA
CTACATGATCGCCGCCGACGGCGCCGGCGGAACGATCCGCGATGTCGGCG
GCGTCAAGATGGAAGGTCCCGGCGTTCTCGCCGATACGATTTCTGTGCTG
TTCGAGGCCGATCTCGACTCGATCCTGCCAGGCGGGGGATTTGCGCTCTA
TTATCTGCGCAACCCGGCGTTCAGCGGCGCCTTCGTCACCTGCGACGAAC
CCAATCACGGCCAGATCAACATCGAATATGATTCCACGCGCGATCAGGCG
TCCGATTTCGACGAAGAACGATGCGAAGCGCTCGTGCGACAATCTCTTGG
CGTCGCCGATCTTGCCGTAAAGATTCTCGATATTCGCCCCTGGCAGATGG
CCGCTCTCCTCGCCGATCGCATGTCTTTCGGGCGGGTGTTTCTCGCGGGC
GACTGCGCGCACATCACGCCGCCCGTTGGCGGGCTCGGCGGACAAACCGC
GATCCAGGACGCCGCCGACCTGGCGTGGAAACTTGCGCTCGTCGTCAAGG
GCCAGGCTGCGCCCACGCTGCTCGACAGCTATGAGATCGAGCGCAGGCCC
GTGGCGCGGATCGCAATCGCGCGGTCCATCGCCAATTACGTCGAGCGCCT
CCTGCCCGATCGTCAGGACATCCGCATTCGGGAAGACGAATACGGCCTGC
TCGAAACTGCTATGGGCTATCGCTACCGCTCCGACGCGATCATCGCTGAT
GAGTTCGACGACGGCGCATGCGTCGAAGATCCTCTTCGCCCGAGCGGCGC
GCCGGGAACGCGTCTGGCGCATGTCTGGTTGCGGCGCGGCGAGGAGACGA
TCTCCTCTCACGATCTGATCGGCCGCGATTTCATGCTTTTTACCGGCCCC
GATGGCGGCGACTGGATCGAGGCGGCGCGGCGCATCGCCCTGCGCTCGAA
AGCGCCGCTTGGCGTCTGCCGCCTCGGCTTCGACGTCGATGATCCCGAAG
GGCTGTTTCTGCCGCGCCTGCGCATTTCGCCTGAAGGCGCGCTCCTCGTG
CGTCCCGACGGCTACATCGCCTGGCGTTCGCGAGGCCGAAGTCCCGATCC
GTTCGCAACGCTCGAAGCGAGTTTTGCGCGCGTGCGCGGTTTCGACACGG
GCCAATCCAGCGGCAGTCACGCGGCTGCGTTCGCCGACGCAAGCTAG
mbnS
(SEQ ID NO: 15)
ATGTCCATGCTGTCGCCGGAGCTCCACGCGCCTTTCCGGTCGGAACCGAT
CCTGGCGGTTGATCCGAAGCGGCTCGAAACGATCCAGGCCCCCTACCAGG
ACTTCAACCGCCTGTCGCGGGCCAGCGGCGCTTTGGAGACCCTGCCCCTT
CGAGCGAAGCGTCAATTCCGCTCCTATTATTTTTTCAATCCGCCAAAATG
GCGCGTCTGGGCGCGTTCGCTCGGCTCCGGCGAACGCATGGCGCCGTCCT
TCGCATCGATCGGCGCGGTGCGCTCCGGCACGTCTTTCCTCTCCTCCTAT
ATTTTTCAGCATCCGCACGTCGTGCTGCCGCTGTCGAAGGAAATCTCCTT
CACGGAGACGATGCGCGAACTCATGGCGCATTTTCCGACGCTTGCGGCGC
AAAGGGCGGCGGAGCGACGCAACGGCGGCGCGATCACCGGCTACTGCACG
CCGGTGATGCCCAATCCACTTTGGATATTTCTGGCGCAGTCGATGTTTCC
AGACATGCGGATCATCTGCGTTCTGCGCGATCCGGTCGAGCGCACTTTTT
CGCACTGGCGATGGGATCAAAAGCGCTTCATCAGCCGCAAGTCGCAGGAC
CCGCATTGGAAGGGCTTTCCGGACCTTCAAACCTTGATCGAAGCCGAAAA
GGCGATAATACGCGGCGGCGGCATGGAGCCGCACGCGTTTTCCGGCGCGC
GGGCCGGCTATCTGCGCCACAGCATCTACGCGCCCTTTCTGCGCCTGCTG
TTCGAGAAATTCGGCAGAGACTGCGTTTTCATCGTCGATGCGGCGGAATT
TTTTCGCGACCCGCGATCGACCGCGAAGGAGATTTACGCGTTTCTCGGCC
TTCCCAACGTCGAGCCGCTGGTGATCGAGGAGCAAAATCCGGGTCCCCCC
GGAACGCTTGAGGACGGACTTCGGGAGAATTTGGCCGAGTTCTTCCGGCC
CTACAATGAAGAGCTCTATGCTCTGCTGGGTAGGGATTTCGGCTGGGGCG
CCGGCCGCTAA
mbnP
(SEQ ID NO: 16)
ATGCGCGCATTCACTCGATTTACCGCCGTTATCGCCGGTCTCGCGGCCAT
TGGCGGCGCGCAGGCGGCGGCGAAGGGCGACGGCGGCGAAAGGCAGCAGG
TCGCGATACGCTTTGCGCTTACCGCGGGCGATCAGCCGGTCGAATGTGGC
CGCGACATTGCCGGGCTAGGAACCGGCGGGCAGCCGGCGCAGCTCCACGA
CGCGAGGTTCTACATCGCCGCGCCGGCGCTGATCGACGCCGCCGGCCACG
AAGTCCCCATCGAACTGGAACAGAACGACTGGCAATACGCCAACCTCGCT
CTGCTCGATTTCGAAAACAAGACCGGCAAATGCGTCGGCAGCGCCGACAT
CAACGACACGATCAAGGGATCGGCGCCGCGCGGCCGTTACAAAGGGTTCA
GCTTCGTCATCGGCGTCCCGAGCGTTGTCCAGGACAAGGACGGCAAGGAC
GTCATTCTCAACCACTCGAATTTCGCCACCGCTCCCGCGCCGCTCGATCT
TCAGTCGATGACGTGGAACTGGCAGGCCGGCCGCAAATTCATAAAGATTG
AAGTTGATCCGGATGGCGGCGTGACCCGACCGCCGCCCCCAGCGAAAAAC
CCCGCCATCGCCACCAGTGGCGCAGGCGAAAAAACTGACGGCCCGCCGCC
GATGGCGGACGCGCCGAAGACGGCGCCGCTGCGCGTCAACTCGGACGGGA
CGATCACCGTCTCGACCTGGATGCTGCACCTTGGTTCGACGGGCTGTCGC
GGCGACGCGACGAAAGGCGAAATCACCTCCTGCGCCAATCCAAATCGCTT
TCCCGTGAAACTCGCGTCCTTCGACCCCGCGCGGCAGCGCGTCGTGCTCG
ATTTGAAGCCGCTGTTCGCCGGCATCGATCTTGGCAAAGACCAGGGATTC
TCGACCGGTTGCATGAGTGGTCCGGCCGATCCGGAATGCACGCCGATGTT
CGAAAATCTCGGGCTACGGCTCAAGGAAACCGCGCCCGACGCCAATGACG
CCGGCCAGTCCTCCGGCGCGTCGACGAAGATTTTCCGCGCGGAGGTCACG
AAATGA
mbnH
(SEQ ID NO: 17)
ATGAAGCGCGAAGCTCTCATTCTCCTCTTCGCCGCAACGCTGATCGCAGG
CGGGGCGCAAGCCGACCCTGTCCCCAGCGCGGAGTCCTGGCGCTGGACTT
TGCCCGACTTCATGCCGCCGCCACGGGTTCCAGCCGACAACGCAATGTCG
GAGGCGCGTTTTCAGCTCGGACGCAAACTCTTCTACGACCCGCGTCTGTC
CGGAAACGGCAAGGAGAGCTGCGCCTCCTGCCATGAGCAACGTCTGGCGT
TTACTGATGGCCGTCCGGTCGCCGTCGGCGCCACGGGCCAGCATACGCCG
CGCAATTCGCCCAGCATCGCCAACGCCGCTTGGCGCGCCACGCTCACCTG
GGCGAATCCGGCGATGGTAACACTGGAAAGGCAGATGGAAGGCCCGCTCT
TTGGCGAAGAGCCGATCGAGATGGGCGTCAATGACAAGAACCGCAAAGAG
ATACTCGCGCGTTTCGACAGTGACGCAGAGTATCGCCGCGACTTCGCGGC
CGCCTTTCCCGGCGAGGCGGACTCGATCAGCTTCGGCAACGTCATCAAGG
CGATCTCCGCTTTCGAGCGCGGCGTCGTCTCGGCGTCCTCGAAATATGAT
CTCTATCTTGAAGGCAAGGCGACGCTCTCTCCAGAAGAGACGCGCGGCAA
GGATCTTTTCTTCGGCGAGAAGGCCGAGTGCCACCACTGCCACGGCAGCG
TGAATTTCGACGATCAGTTCTATCACGCCAAAACGCGCGAGATCGAGACG
CCGTTTCACAACACCGGCCTCTATGATCTCGATGGCAAAGGCGCCTATCC
CGAGCCCAATCGCGGCGTTTTCGAGAACACGCATAATCCCGCCGAGATGG
GCGCGTTTCGCGCGCCGAGCCTGCGCAATATAGCTGTCACCGCGCCTTAC
ATGCACGACGGCAGCGTCGCGACGCTCGATGACGTGATCGACATTTACGC
GGCCGGCGGACGCAATGTCACGAGCGGTCCACTCAAGGGCGACGGTCGCT
ACAACGCAAACAAAAGCGCGTTGATCGTTCCCATCGATCTGTCGCCTCAG
GAAAAGAGCGATCTGATCGCCTTTCTGAAAACGCTGACCGACGAGTCGTT
GCTGACATCTCCACGTTTCGCCGACCCCTGGAAACCGGCGTCGACGGCAA
GCGGGCCATCGCGTGCGTCCTCCCATTGA

In some embodiments, heterologous expression of one or more genes from a methanobactin gene cluster in a different species indicates an insertion of one or more sequences each having at least 80% identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to one or more of SEQ ID NO: 10-17 into the genome of the mutant organism, such that the mutant organism expresses the heterologous gene. For example, heterologous expression of mbnA indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 10 is inserted and thus heterologously expressed in the mutant organism, heterologous expression of mbnB indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 11 is inserted and thus heterologously expressed in the mutant organism, heterologous expression of mbnC indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 12 is inserted and thus heterologously expressed in the mutant organism, heterologous expression of mbnM indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 13 is inserted and thus heterologously expressed in the mutant organism, heterologous expression of mbnF indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 14 is inserted and thus heterologously expressed in the mutant organism, heterologous expression of mbnS indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 15 is inserted and thus heterologously expressed in the mutant organism, heterologous expression of mbnP indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 16 is inserted and thus heterologously expressed in the mutant organism, and heterologous expression of mbnH indicates that a sequence having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity) to SEQ ID NO: 17 is inserted and thus heterologously expressed in the mutant organism. In some embodiments, one or more sequences (e.g. one or more sequences, two or more sequences, three or more sequences, four or more sequences, five or more sequences) each having at least 80% sequence identity to one or more of SEQ ID NO: 3-9 are deleted and one or more sequences (e.g. one or more sequences, two or more sequences, three or more sequences, four or more sequences, five or more sequences) each having at least 80% sequence identity to one or more of SEQ ID NO: 10-17 are inserted in the mutant organism.

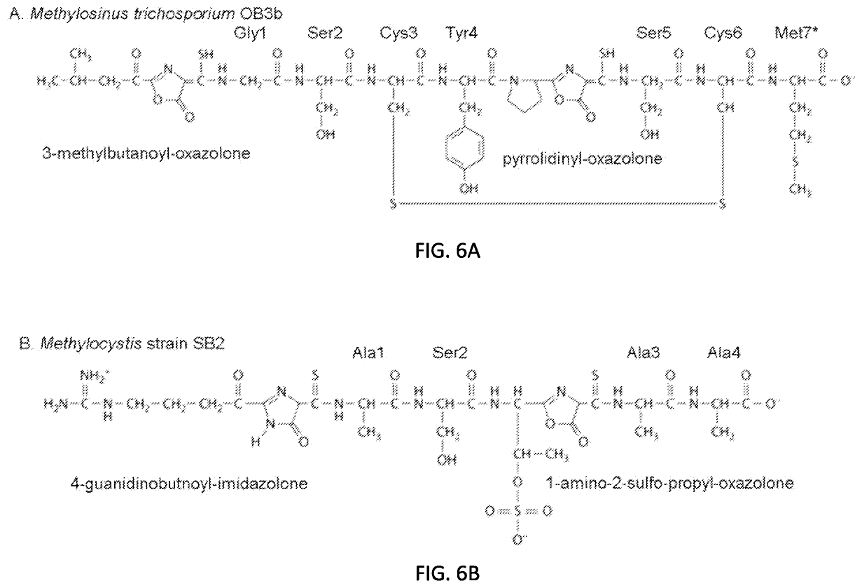

In some embodiments, the mutant organism is a Methylosinus species and the genes heterologously expressed in the mutant organism are from a Methylocystis species. For example, in some embodiments it may be advantageous to use a Methylosinus species (e.g. Methylosinus trichosporium OB3b) as the mutant organism due to ease and convenience of growing the organism, while heterologously expressing methanobactin genes (e.g. one or more genes from a methanobactin gene cluster) from a Methylocystis species that produces a group II methanobactin. In some embodiments, the mutant organism is a Methylorubrum species and the genes heterologously expressed in the mutant organism are from a Methylocystis species. For example, in some embodiments the mutant organism is Methylorubrum extorquens. In some embodiments, the heterologously expressed methanobactin genes are from Methylocystis strain SB2, which produces the highly potent copper chelator methanobactin mb-SB2. The formula of methanobactin SB2 is shown in FIG. 6B. Methanobactin SB2, along with medical uses for methanobactins, are disclosed in U.S. Patent Publication No. US20210369809A1 and WO2022162232A1, the entire contents of each of which are incorporated herein by reference for all purposes.

In some embodiments, the mutant organism is Methylosinus trichosporium OB3b, the expression of one or more native genes in a methanobactin gene cluster is attenuated in the organism, and one or more genes from a gene cluster from Methylocystis sp. strain SB2 are heterologously expressed in the mutant organism. In some embodiments, the mutant organism is Methylosinus trichosporium OB3b, the expression of at least mbnA, mbnB, mbnC, and mbnM is attenuated in the organism and replaced by one or more genes in a methanobactin gene cluster from Methylocystis sp. strain SB2 which are heterologously expressed in the mutant organism.

In some embodiments, the mutant organism is Methylorubrum extorquens, the expression of one or more native genes in a methanobactin gene cluster is attenuated in the organism, and one or more genes from a gene cluster from Methylocystis strain SB2 are heterologously expressed in the mutant organism. In some embodiments, the mutant organism is Methylorubrum extorquens, the expression of at least mbnA, mbnB, mbnC, and mbnM is attenuated in the organism and replaced by one or more genes in a methanobactin gene cluster from Methylocystis strain SB2 which are heterologously expressed in the mutant organism.

In some aspects, provided herein are mutant organisms wherein the gene sequence of mbnA is modified to ultimately produce smaller forms of methanobactin. For example, in some embodiments provided herein are mutant organisms expressing a modified mbnA gene, wherein the modified mbnA gene comprises one or more mutations such that the resulting mature methanobactin produced by the organism (e.g. produced at least in part by expression of the mutated mbnA gene) is smaller than the wildtype (e.g. unmodified) form of methanobactin. For example, the gene sequence of mbnA can be modified such that one or more C-terminal amino acids are removed from the resulting methanobactin. As another example, the gene sequence of mbnA can be modified such that one or more amino acids between the two rings (e.g. one or more of the 2-5 amino acids between the oxazolone ring and the oxazolone, imidazolone, or pyrazinedione ring) are missing from the resulting methanobactin produced following gene expression. In some embodiments, smaller forms of methanobactin may be sufficiently small to penetrate the blood brain barrier and be used to extract excess copper from the brain.

The mutant organisms provided herein find use in methods of producing methanobactin. The methods may comprise culturing the mutant organism under suitable conditions such that the mutant organism produces methanobactin. In some embodiments, the mutant organism is cultured in the presence of methanol. In some embodiments, provided herein is a method of producing methanobactin comprising culturing a mutant organism provided herein in the presence of methanol, such that methanobactin is produced. In some embodiments, provided herein is a method of producing methanobactin comprising culturing a mutant organism provided herein in the presence of methanol and copper (e.g. CuCl₂), such that methanobactin is produced.

Suitable conditions for culturing the mutant organisms such that methanobactin is produced are described in US20120034594A1, the entire contents of which are incorporated herein by reference for all purposes. The precise culture conditions will vary depending on the strain of bacterium used. In some embodiments, the culture is a liquid culture, e.g., the culture is maintained in a liquid medium. In some embodiments, the culture is a solid culture, e.g., the culture is maintained on a solid medium. In some embodiments, the mutant organisms are cultured in an environment comprising nutrients essential to the growth of the cell, such as a nitrogen source (e.g., dinitrogen, nitrate, ammonium, L-isoleucine, L-proline, L-glutamine), and a carbon source (e.g., methane, ethanol, acetate). In some embodiments, the carbon source is methanol. In some embodiments, the mutant organisms are cultured in a medium comprising copper (e.g., medium comprising CuCl₂). In some embodiments, the mutant organisms are cultured in medium lacking copper (e.g., medium lacking CuCl₂). In some embodiments, the mutant organisms are cultured at a pH between 6 and 9. For example, in some embodiments the mutant organisms are cultured at pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6, pH 8.7, pH 8.8, pH 8.9, and pH 9.0. In some aspects, the mutant organisms are cultured at a temperature between about 10° C. and about 37° C. In some embodiments, the mutant organisms are cultured at a temperature of between about 10° C. and about 37° C., between about 10° C. and about 36° C., between about 10° C. and about 35° C., between about 10° C. and about 34° C., between about 10° C. and about 33° C., between about 10° C. and about 32° C., between about 10° C. and about 31° C., or between about 10° C. and about 30° C.

In some embodiments, the mutant organism produces at least 50% more methanobactin compared to a wildtype form of the organism when cultured in the presence of methanol. In some embodiments, the mutant organism produces at least 50% more methanobactin compared to a wildtype form of the organism when cultured in the presence of methanol and copper. For example, in some embodiments the mutant organism produces at least 50% more, at least 55% more, at least 60% more, at least 65% more, at least 70% more, at least 75% more, at least 80% more, at least 85% more, at least 90% more, at least 95% more, at least 100% more, or more than 100% more (e.g. more than double the amount) methanobactin compared to a wildtype form of the organism. Accordingly, the mutant organisms and methods of use thereof provided herein enable safe, large-scale production of methanobactin using methanol, rather than methane, as an energy source.

In some embodiments, the methanobactin produced by the mutant organisms described herein is isolated following production. In some embodiments, the methanobactin is modified, such as by making one or more chemical modifications. Suitable methods for isolating methanobactins include, for example, fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography. Exemplary methods for isolating and for modifying (e.g. chemical modifications) methanobactins are described in WO2022162232A1, U.S. Pat. No. 8,735,538B1, and U.S. Pat. No. 7,932,052B1 the entire contents of each of which are incorporated herein by reference for all purposes.

### EXAMPLES

### Example 1

ΔmmoD Mutant

Transition metals such as copper are key cofactors in a variety of metabolic processes, and play an important role in the development of healthy nerves, bones, collagen, and melanin. However, defective regulation of copper levels, such as defective copper transport mechanisms, results in cellular damage. Various disorders related to defective regulation of copper levels have been identified, including Menkes Disease, occipital horn syndrome, and Wilsons Disease. Currently approved copper chelators used for treatment of copper-related disorders are known to have adverse effects, including bone marrow toxicity, nephrotoxicity, hepatotoxicity, anemia, and triggering of autoimmune disease. Accordingly, improved agents for treatment of copper-related disorders are greatly needed.

Methanobactin has recently emerged as a potential therapeutic agent for the treatment of Wilson's disease and other copper-related disorders. Methanobactin is a copper chelating molecule produced by some methanotrophs and some methylotrophs. Methanobactin thus represents a natural product involved in copper homeostasis in these organisms. However, mass production of methanobactin for potential therapeutic use is limited, as current strategies rely on methane-based fermentation technologies that pose multiple challenges, including inadequate production of methanobactin due to natural inhibitory feedback mechanisms in the organisms used for production, and the explosive nature of methane: air mixtures. As such, improved methods for mass production of methanobactins are needed.

The present disclosure provides mutant organisms with greatly enhanced methanobactin production. The organisms can produce methanobactin in the presence of either methane or methanol. Thus, the methylotrophic organisms described herein are advantageous over methanotrophs, as methanol is a much safer energy source compared to methane.

The first step for methane metabolism is the oxidation of methane to methanol. The responsible enzyme for this step/reaction is methane monooxygenase (MMO). There are two types of MMOs—a membrane-bound or particulate MMO (pMMO) and a cytoplasmic or soluble MMO (sMMO). pMMO is a copper/iron-containing enzyme and its activity is strongly dependent on copper. sMMO is an iron-containing enzyme with a diiron center at its active site. Some methanotrophs, e.g., Methylosinus trichosporium OB3b and Methylococcus capsulatus Bath, can express both pMMO and sMMO. In these methanotrophs, the expression and activity of the two forms of MMO is controlled according to copper availability, i.e., the “copper switch”. That is, in those microbes that can express both forms of MMO, sMMO expression and activity is only observed in the absence of copper, while in the presence of copper, sMMO expression is repressed and pMMO expression and activity increases with increasing copper. sMMO has three components encoded by the mmo gene cluster: the hydroxylase (MMOH) encoded by mmoXYZ, a reductase encoded by mmoC, and an electron shuttle protein encoded by mmoB (10, 17) (FIG. 1). To initialize the catalysis, two electrons are delivered from MmoC to the diiron center of MMOH, reducing the diiron center from a diferric state to a diferrous state. MmoB binds to MMOH and mediates the transfer of electrons to the active site of MMOH where methane and oxygen are bound. In addition to these polypeptides, MmoD is also encoded in the mmo gene cluster (FIG. 1). MmoD is not involved in the catalysis of sMMO and plays a role in regulating gene expression.

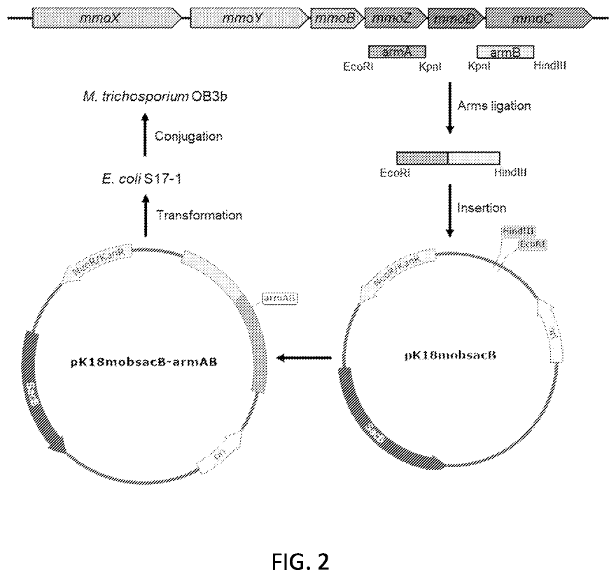

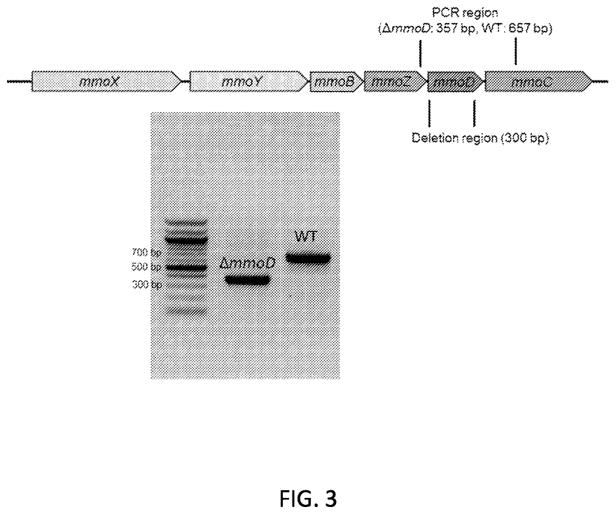

Construction of Methylosinus trichosporium OB3b ΔmmoD mutant mmoD was knocked out in the MB-producing methanotroph Methylosinus trichosporium OB3b. FIG. 2 shows a schematic representation of the construction of M. trichosporium OB3b ΔmmoD mutant. Briefly, upstream and downstream regions of mmoD (arms A and B, respectively) were amplified using the primers listed in Table 1. Arms A and B were digested with the appropriate restriction enzymes, and ligated together to form armAB which was subsequently inserted into pK18mobsacB mobilizable suicide vector (Schafer A, Tauch A, Jager W, Kalinowski J, Thierbach G, Pühler A. 1994. Small mobilizable multipurpose cloning vectors derived from the Escherichia coli plasmids pK18 and pK19: selection of defined deletions in the chromosome of Corynebacterium glutamicum. Gene 145:69-73). pK18mobsacB vector with armAB was transferred to E. coli Top10 (Invitrogen, Carlsbad, CA). The plasmid was then extracted from transformed E. coli Top10 using the Plasmid Mini Kit (Qiagen, Hilden, Germany) following the manufacturer's instructions. The extracted plasmid was then transferred to E. coli S17-1 (Simon R. 1984. High frequency mobilization of gram-negative bacterial replicons by the in vitro constructed Tn 5-Mob transposon. Mol Gen Genet 196:413-420). Conjugation of E. coli S17-1 carrying the constructed vector with M. trichosporium OB3b was performed as described previously (Martin H, Murrell J. 1995. Methane monooxygenase mutants of Methylosinus trichosporium constructed by marker-exchange mutagenesis. FEMS Microbiol Lett 127:243-248). Transconjugants were grown on NMS plates supplemented with 25 μg/ml kanamycin and 10 μg/ml nalidixic acid. One kanamycin resistant transconjugant colony (generated after 10 days of incubation) was transferred to an NMS plate with kanamycin (25 μg/ml) and incubated for 7 days, and subsequently transferred to an NMS plate with 2.5% sucrose (mass/vol). Sucrose-resistant colonies were generated after 10 days incubation and were screened for deletion of mmoD by colony PCR. Successful mmoD deletion mutant was further confirmed by PCR with DNA extracted from the mutant using the DNeasy PowerSoil Pro Kit (Qiagen, Hilden, Germany) following manufacturer's instructions. Results (e.g. confirmation of deletion) are shown in FIG. 3.

TABLE 1
Primers used in this study

| Primer name | Sequence (5′-3′)^{a} |
|---|---|
| mmoD-armA_F | ATTTTT gaattc |
| | (SEQ ID NO: 18) |
| | GGCACCAAATTCACCATCACC^{b} |
| | (SEQ ID NO: 19) |
| | ATTTTT ggtacc |
| | (SEQ ID NO: 20) |
| mmoD-armA_R | GAACGCATGTCTTTCGCAAT |
| | (SEQ ID NO: 21) |
| | ATTTTT ggtacc |
| | TGCTGCGCGAGATCGGAA^{b} |
| | (SEQ ID NO: 22) |
| mmoD-armB_F | ATTTTT aagctt |
| | (SEQ ID NO: 23) |
| mmoD-armB_R | GACGGATGAAGAATTCGAGCTG |
| | (SEQ ID NO: 24) |

^{a}Y, S, and R are the IUPAC DNA codes for the C/T, C/G, and A/G nucleobases, respectively
^{b}Lowercase letters indicate EcoRI, KpnI, or HindIII restriction site sequences included in these primers
^{c}Targeting region indicated in FIG. 2

### RNA Isolation and Reverse Transcription-Quantitative PCR (RT-qPCR)

RNA isolation was performed with a bead-beating procedure followed by column purification using an RNeasy Mini Kit (Qiagen, Hilden, Germany). Genomic DNA was removed from the column with RNase-free DNase (Qiagen, Hilden, Germany) treatment. Absence of genomic DNA was confirmed by 16S rRNA gene targeted PCR with extracted RNA samples as templates. Purified RNA was quantified using a NanoDrop 1000 Spectrophotometer (Thermo Scientific, Wilmington, DE). cDNA was synthesized from 200 ng total RNA using SuperScript™ III Reverse Transcriptase (Invitrogen, Carlsbad, CA) following manufacturer's instructions.

RT-qPCR was performed to determine the relative expression of the mbnA in M. trichosporium OB3b and ΔmmoD mutant grown in presence or absence of copper. RT-qPCR was performed using the iTaq Universal SYBR Green Supermix (Bio-Rad, Hercules, CA) with 96-well PCR plates on a CFX Connect real-time PCR detection system (Bio-Rad, Hercules, CA). The RT-qPCR program was: 95° C. for 10 min, followed by 40 cycles of 95° C. for 15 s, 56° C. for 30 s and 72° C. for 30 s. Melting curves were measured from 65° C. to 95° C. with increments of 0.5° C. and 10 s at each step. Transcription of the targeted genes was determined using cDNA as the template. The transcript levels were calculated by relative quantification using the 2-ΔΔCq method (Schmittgen T D, Livak K J. 2008. Analyzing real-time PCR data by the comparative CT method. Nat Protoc 3:1101-1108) with the 16S rRNA gene as the reference gene (Kalidass B, Ul-Haque M F, Baral B S, DiSpirito A A, Semrau J D. 2015. Competition between metals for binding to methanobactin enables expression of soluble methane monooxygenase in the presence of copper. Appl Environ Microbiol 81:1024-1031). RT-qPCR primers targeting mbnA and 16S rRNA gene of M. trichosporium OB3b were obtained from former studies (Gu W, Baral B S, DiSpirito A A, Semrau J D. 2017. An aminotransferase is responsible for the deamination of the N-terminal leucine and required for formation of oxazolone ring A in methanobactin of Methylosinus trichosporium OB3b. Appl Environ Microbiol 83:e02619-16; Muyzer G, De Waal E C, Uitterlinden A G. 1993. Profiling of complex microbial populations by denaturing gradient gel electrophoresis analysis of polymerase chain reaction-amplified genes coding for 16S rRNA. Appl Environ Microbiol 59:695-700).

### Methanobactin (MB) Analysis

MB production was measured in M. trichosporium OB3b wild type and ΔmmoD mutant cultures grown with methane and methanol. 1 ml culture was collected at the stationary growth phase and centrifuged at 13,000 rpm for 1 min. The supernatant was transferred and analyzed on an UV-visible spectroscopy at wavelength 250-500 nm.

### Results

RT-qPCR and UV-Vis Analyses of MB Expression and Production in M. trichosporium OB3b Wild Type and ΔmmoD Mutant

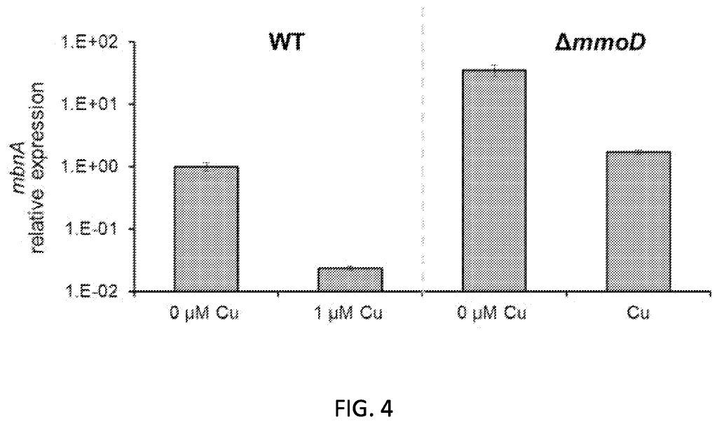

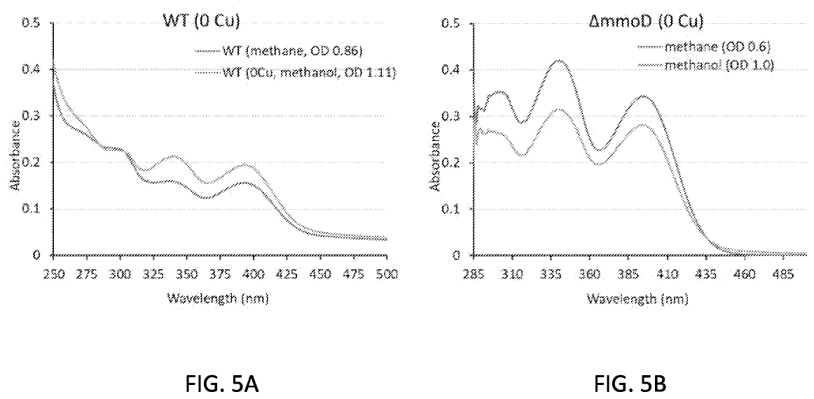

In the absence of copper, expression of the putative methanobactin precursor peptide mbnA in ΔmmoD mutant was ~35-fold higher than the wild type M. trichosporium OB3b when grown on methane. In the presence of 1 μM copper, mbnA expression was ~200-fold higher in ΔmmoD mutant than the wild type M. trichosporium OB3b (FIG. 4). UV-Vis spectroscopy showed characteristic peaks (325-350 and 375-425 nm) of MB in the supernatant of M. trichosporium OB3b wild type and ΔmmoD mutant cultures (FIG. 5). UV absorption was remarkably increased in ΔmmoD mutant culture compared to wild type M. trichosporium OB3b, indicating a much higher MB production in ΔmmoD mutant than wild type M. trichosporium OB3b. Notably, the methanol grown culture of ΔmmoD mutant also showed production of MB, and the amount was significantly higher than that of wild type M. trichosporium OB3b grown with either methane or methanol (FIG. 5).

### Conclusions

M. trichosporium OB3b ΔmmoD mutant produce MB with either methane or methanol as a growth substrate, and the MB producing amount is significantly higher than wild type M. trichosporium OB3b.

### Example 2

### Additional Mutants

Aerobic methanotrophs, or methane-consuming microbes, are strongly dependent on copper for their activity. To satisfy this requirement, some methanotrophs produce a copper-binding compound, or chalkophore, called methanobactin (MB). In addition to playing a critical role in methanotrophy, MB has also been shown to have great promise in treating copper-related human diseases, perhaps most significantly Wilson Disease. In this congenital disorder, copper builds up in the liver, leading to irreversible damage and, in severe cases, complete organ failure. Remarkably, MB has been shown to reverse such damage in animal models, and there is a great interest in upscaling MB production for expanded clinical trials. Such efforts, however, are currently hampered as: (1) the natural rate of MB production rate by methanotrophs is low, (2) the use of methane as a substrate for MB production is problematic as it is explosive in air, (3) there is limited understanding of the entire pathway of MB biosynthesis, and, (4) the most attractive form of MB is produced by Methylocystis sp. strain SB2, a methanotroph that is genetically intractable. Herein we report heterologous biosynthesis of MB from Methylocystis sp. strain SB2 in an alternative methanotroph—Methylosinus trichosporium OB3b—not only on methane but methanol as well.

Methanobactin (MB) is secreted by some aerobic methanotrophs for copper sequestration, i.e., MB is a copper-binding compound or “chalkophore” analogous to siderophores produced by many organisms for iron collection. MBs are small (<1,350 Da) ribosomally synthesized post-translationally modified polypeptides (RiPPs) that have high affinity and specificity for copper (~10²⁰-10³⁰ M⁻¹).

To date, all identified MBs can be divided into two general groups based on structure and genetic organization—Group I and II. Thus far, four Group I MBs have been characterized, with the first and studied Group I MB isolated from Methylosinus trichosporium OB3b (MB-OB3b) (FIG. 6A). Group I MBs are distinguished by the presence of two heterocylic rings—an oxazolone ring at the C-terminus, and, either another oxazolone ring at the N-terminus, or in some cases, a pyrazinedione ring. Group I MBs include a disulfide bridge between two cysteine residues (FIG. 6A). Copper is chelated by the N- and S-ligands of the heterocyclic rings and thioamides, respectively, causing MB to form a pyramid-like shape after binding copper.

Four Group II MBs have also been characterized to date, with the first examined MB being that from Methylocystis sp. strain SB2 (MB-SB2; FIG. 6B). Group II MBs, like Group I MBs, have two heterocyclic rings with the C-terminal ring being an oxazolone ring. Unlike Group I MBs, however, the N-terminal ring has not been found to be another oxazolone ring; rather it is either an imidazolone ring (as shown in FIG. 6B for MB-SB2), or a pyrazinedione ring. Further, the disulfide bridge found in Group I MBs is absent from Group II MBs, and instead these MBs are sulfonated. Evidence indicates that the addition of a sulfate group adjacent to the C-terminal oxazolone ring serves to enhance copper binding, and also allows Group II MBs to form a hairpin-like structure after binding copper via the N-ligand of the oxazolone/imidazolone and S-ligand of thioamides.

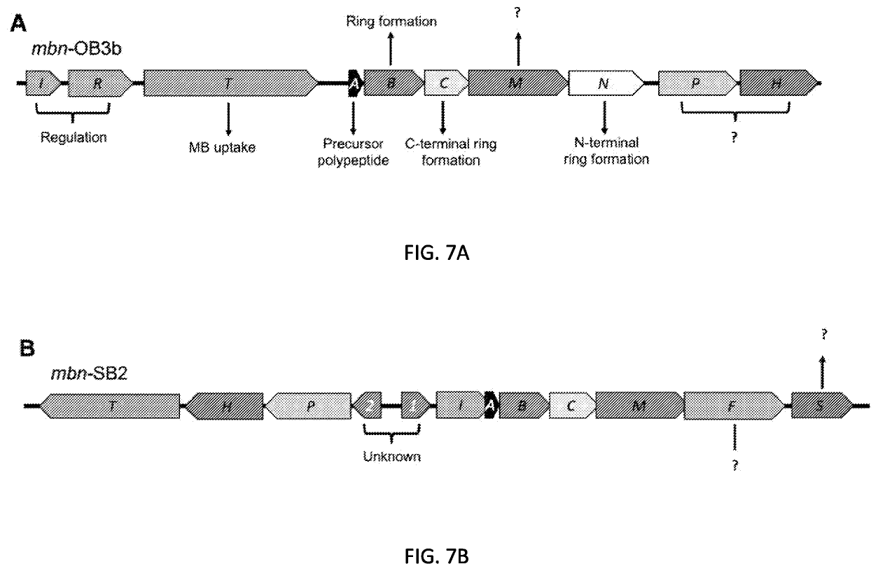

The genetics and biochemistry of Group I MBs have been evaluated, largely through the examination of MB from of M. trichosporium OB3b as this strain is genetically tractable. From a suite of knock-out assays, it has been shown that: (1) the MB precursor polypeptide is encoded by mbnA, (2) formation of the N-terminal oxazolone ring of MB-OB3b requires activity of a specific aminotransferase encoded by mnbnN, (3) formation of the C-terminal oxazolone ring involves the activity of the gene product of mbnC, (4) uptake of MB-OB3b requires a TonB-dependent transporter encoded by mbnT, and, (5) mbnI and mbnR play key roles in controlling mbn gene expression (FIG. 7A). The roles of mbnM, mbnH, and mbnP are not certain.

The genetics and biochemistry of Group II MB are not well-characterized, largely due to the fact that none of the methanotrophs shown to make this form of MB are genetically tractable. The roles of mbnF, mnnS, mbnP, and mbnI, for example, are unclear. (FIG. 7B).

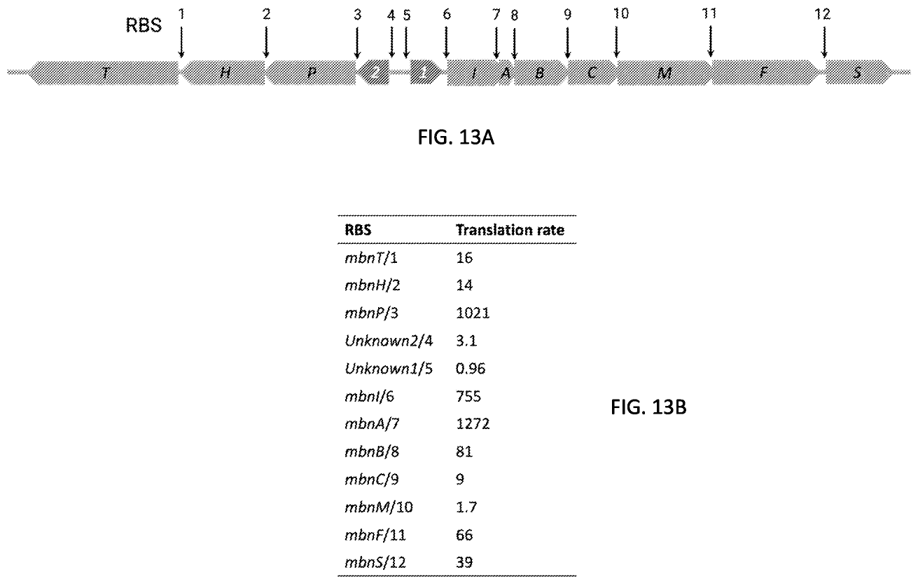

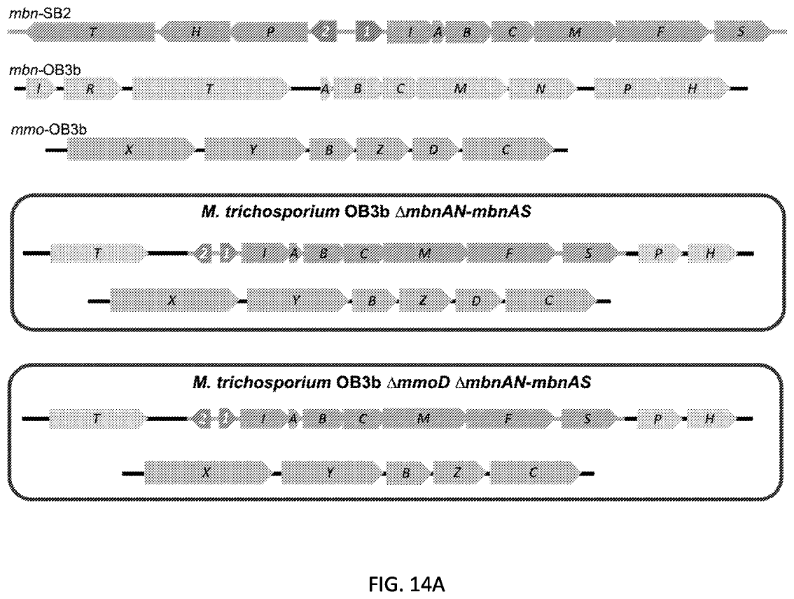

Bioinformatic analysis of the mbn gene cluster of Methylocystis sp. strain SB2 Bioinformatic analyses of the mbn gene cluster Methylocystis sp. strain SB2 identified three strong promoters upstream of nbnI, mbnP, and mbnH (FIG. 7, 12). A ribosome binding site (RBS) was detected for each gene of the mbn gene cluster and the predicted translation rate of mbnI, mbnA, and mbnP was significantly higher (10- to 750-fold) than other genes of the putative methanobactin gene cluster of Methylocystis sp. strain SB2 (FIG. 13). Collectively these data suggest that all of these genes, including the unknown genes, may have roles in MB-SB2 biosynthesis.

Knock-In and Expression of mbnAS of Methylocystis sp. SB2 in M. trichosporium OB3b ΔmbnAN and ΔmmoD

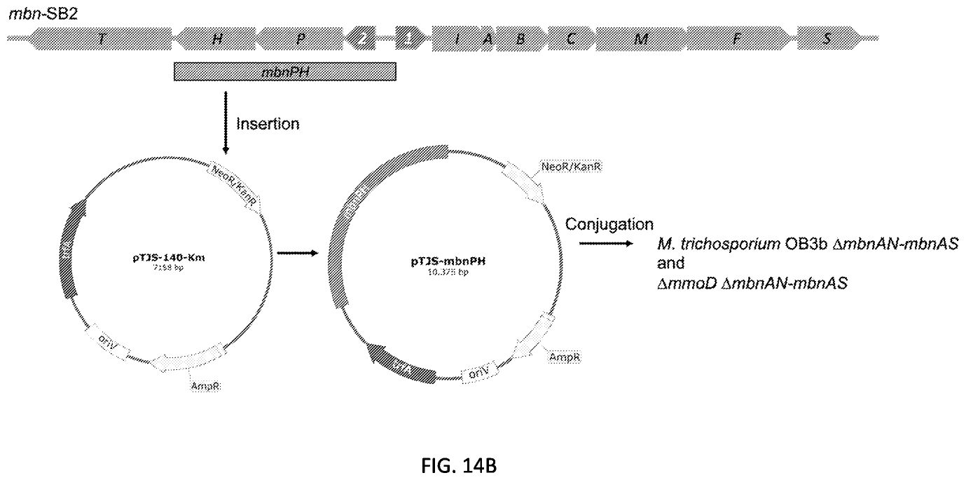

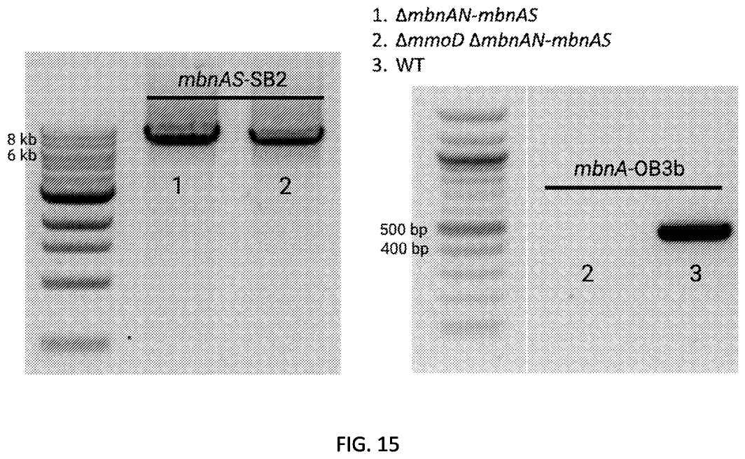

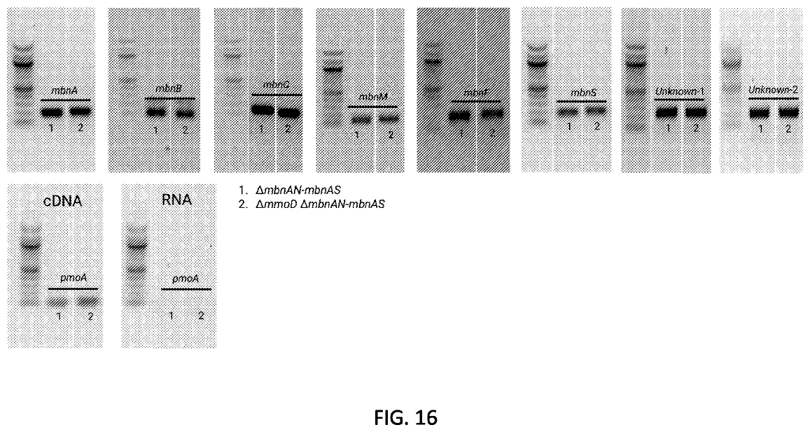

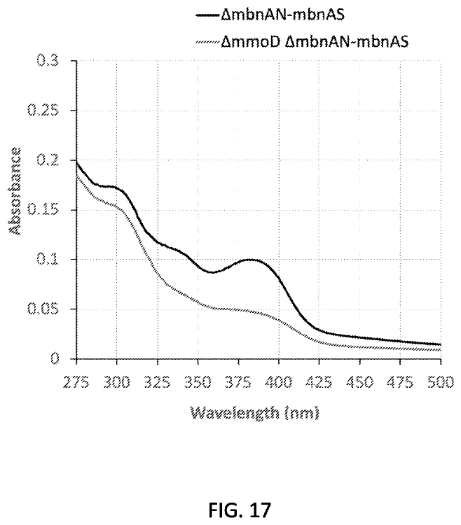

To investigate if MB-SB2 can be heterologously synthesized in M. trichosporium OB3b, the two genes of unknown function and mbnABCMFS of Methylocystis sp. strain SB2 (hereafter termed mbnAS), were inserted into the chromosome of two strains of M. trichosporium OB3b—one where native mbn genes of M. trichosporium OB3b were removed (mbnABCMN, or ΔmbnAN+mbnAS), and another where mmoD was also removed (ΔmbnAN ΔmmoD+mbnAS, FIG. 14A). The presence of mbnAS from Methylocystis sp. strain SB2 as well as the absence of native mbn genes (i.e., mbnABCMN) in the chromosome of these two strains were verified by PCR (FIG. 15) and sequencing (data not shown). Transcriptional analysis showed that all knocked-in genes were expressed in both M. trichosporium OB3b ΔmbnAN+mbnAS and ΔmmoD ΔmbnAN+mbnAS (FIG. 16). However, UV-Vis measurement of the supernatant of these strains actively growing on methane suggests that any MB produced was in low quantity and incomplete. That is, only a small absorption peak at 386 nm was observed for either strain, suggesting that the N-terminal imidazolone ring of MB-SB2 was present, but in low amounts. There was no evidence of any absorption peak at 338 nm, indicating the C-terminal oxazolone ring of MB-SB2 was not formed (FIG. 17). Further efforts to purify MB from either strain was unsuccessful (data not shown).

Expression of mbnPH of Methylocystis sp. Strain S B2 in ΔmbnAN-mbnAS and ΔmmoD ΔmbnAN-mbnAS and their MB-SB2 Production

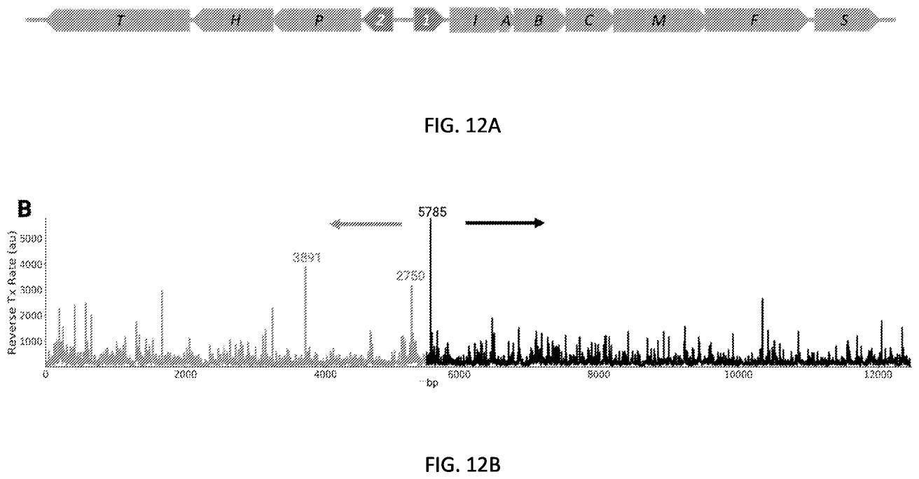

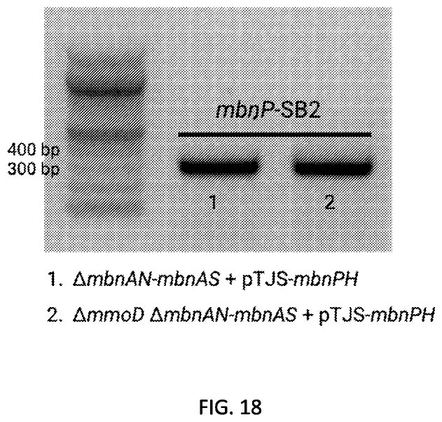

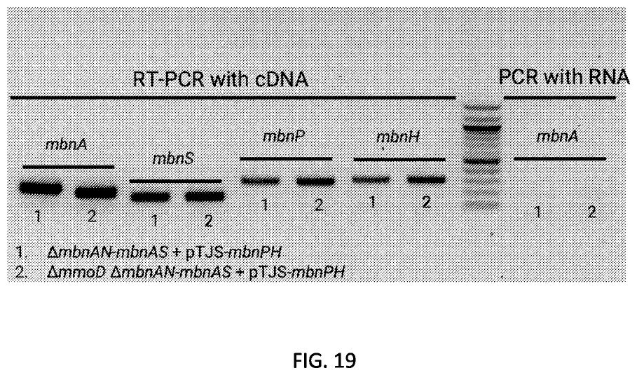

As little MB-SB2 was found to be produced when the genes were expressed heterologously in mutants of M. trichosporium OB3b unable to produce MB-OB3b, it was speculated that additional gene(s) is(are) needed, i.e., mbnPH. Initially it was assumed that these genes, although commonly found adjacent to genes critical for MB biosynthesis, were not needed for two reasons: (1) it has been widely assumed that these genes have no role in MB biosynthesis (Kenney G E, Rosenzweig A C. Chalkophores. Ann Rev Biochem 2018; 87:645-76), and (2) the native mbnPH genes of M. trichosporium OB3b were not removed and are very similar to mbnPH of Methylocystis sp. strain SB2 (E values of 10⁻⁷⁷ and 10⁻¹⁶⁷ for mbnP and mbnH, respectively). Further investigation, however, suggested that these genes may be important as in silico analyses predict that the promoter for mbnPH of Methylocystis sp. strain SB2 is particularly strong (FIG. 12), as well as mbnP having a high translation rate (FIG. 13). To consider this, a plasmid based on pTJS-140 (Martin, H.; Murrell, J. Methane monooxygenase mutants of Methylosinus trichosporium constructed by marker-exchange mutagenesis. FEMS Microbiol. Lett. 1995, 127 (3), 243-248) was constructed with mbnPH inserted and transferred to M. trichosporium OB3b ΔmbnAN+mbnAS and ΔmmoD ΔmbnAN+mbnAS for expression of Methylocystis sp. strain SB2 mbnPH. The presence of mbnP in ΔmbnAN-mbnAS+pTJS-mbnPH and ΔmmoD ΔmbnAN-mbnAS+pTJS-mbnPH was verified by PCR (FIG. 18). Transcriptional analysis also showed that mbnPH were expressed in both ΔmbnAN+mbnAS+pTJS-mbnPH and ΔmmoD ΔmbnAN+mbnAS+pTJS-mbnPH (FIG. 19).

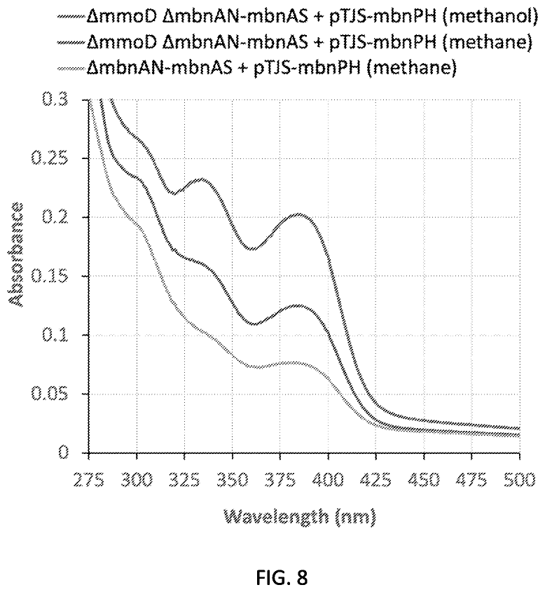

UV-Vis absorption spectrophotometry of the spent medium of these constructs clearly showed evidence of MB-SB2 production (FIG. 8), where characteristic peaks at wavelengths of 303, 336, and 386 nm were observed (the latter two associated with the oxazolone and imidazole rings of MB-SB2, respectively). Overall, absorption was higher in the supernatant of ΔmmoD ΔmbnAN+mbnAS+pTJS-mbnPH than ΔmbnAN-mbnAS+pTJS-mbnPH, indicating higher MB-SB2 production in ΔmmoD ΔmbnAN-mbnAS+pTJS-mbnPH. This construct was thus selected for further investigation. Moreover, it was found that ΔmmoD ΔmbnAN-mbnAS+pTJS-mbnPH could also grow with methanol, and that MB-SB2 production in this strain was culture was significantly higher when methanol was used as the growth substrate as compared to methane-grown cultures (FIG. 8).

### Verification of Heterologous Production of MB-SB2

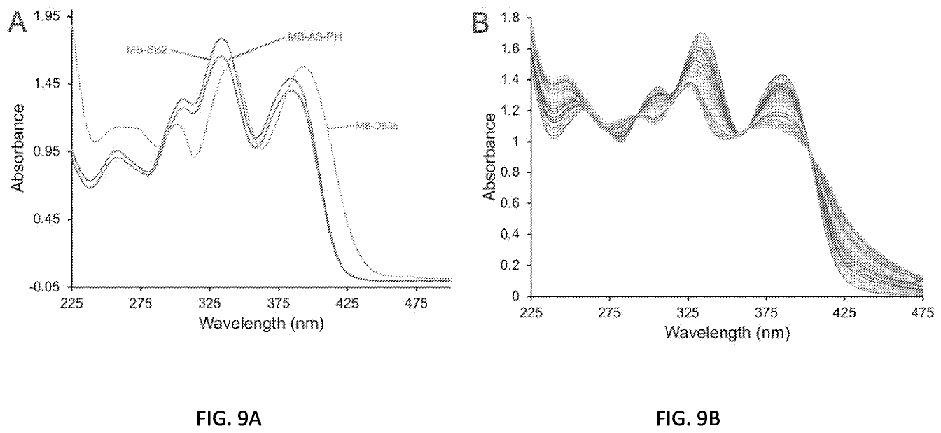

To confirm that SB2-MB was heterologously produced using M. trichosporium OB3b as a production platform, the ΔmmoD ΔmbnAN-mbnAS+pTJS-mbnPH construct was grown in a 10-liter fermenter and MB purified from the spent medium. Comparison of the UV-Visible absorption spectra of MB from ΔmmoD ΔmbnAN-mbnAS+pTJS-mbnPH to that of verified MB-OB3b and MB-SB2 standards clearly shows that the MB made by this production construct closely resembles that of SB2-MB (FIG. 9A). In addition, copper titration assays show the same pattern of absorption maxima decrease with increasing copper, as well as a slight blue shift of the peak at 338 nm (diagnostic for a C-terminal oxazolone ring) to 326 nm as previously observed for MB-SB2 (Krentz, B. D.; Mulheron, H. J.; Semrau, J. D.; DiSpirito, A. A.; Bandow, N. L.; Haft, D. H.; Vuilleumier, S.; Murrell, J. C.; McEllistrem, M. T.; Hartsel, S. C. A comparison of methanobactins from Methylosinus trichosporium OB3b and Methylocystis strain SB2 predicts methanobactins are synthesized from diverse peptide precursors modified to create a common core for binding and reducing copper ions. Biochemistry 2010, 49 (47), 10117-10130; Bandow, N.; Gilles, V. S.; Freesmeier, B.; Semrau, J. D.; Krentz, B.; Gallagher, W.; McEllistrem, M. T.; Hartsel, S. C.; Choi, D. W.; Hargrove, M. S. Spectral and copper binding properties of methanobactin from the facultative methanotroph Methylocystis strain SB2. J. Inorg. Biochem. 2012, 110, 72-82). (FIG. 9B).

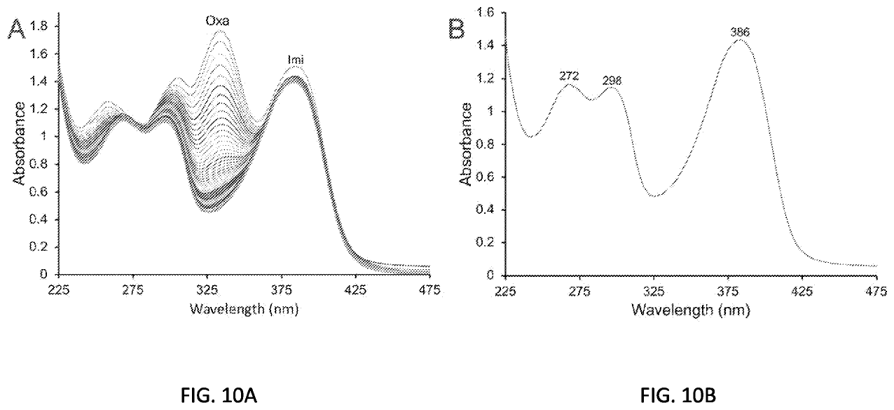

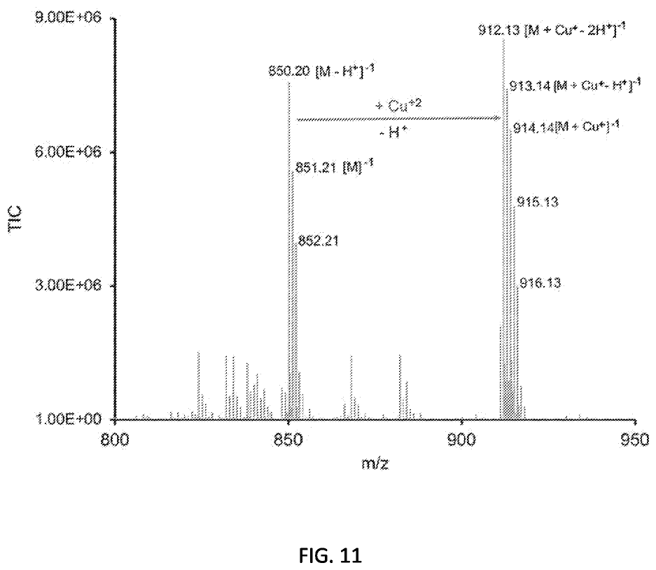

In addition to copper titration assays, the purified MB from ΔmmoD ΔmbnAN-mbnAS+pTJS-mbnPH was subjected to acid digestion. The oxazolone rings of MB are particularly susceptible to acid-catalyzed hydrolysis while the N-terminal imidazolone ring of MB-SB2 is very resistant to such degradation. Indeed, digestion of MB from ΔmmoD ΔmbnAN-mbnAS+pTJS-mbnPH in 100 μM acetic acid over 10 hours clearly shows complete loss of the C-terminal oxazolone ring as indicated by the loss of the absorption peak at 338 nm. The absorption peak at 387 nm, however, showed little decrease, and thus is indicative that this peak is indeed not due to an oxazolone ring, but rather results from the N-terminal ring being an imidazolone moiety (FIG. 10). Furthermore, the UV-visible absorption spectra of the MB from ΔmmoD ΔmbnAN-mbnAS+pTJS-mbnPH following hydrolysis of the oxazolone group was identical to that observed following hydrolysis of the oxazolone group in MB-SB2 (FIG. 10B).

Mass Spectrometry of MB-SB2 with and without Copper

Metal-free MB-SB2 produced by M. trichosporium OB3b ΔmmoD ΔmbnAN-mbnAS+pTJS-mbnPH was showed a molecular mass of 851.21 (FIG. 11) which was identical to what was originally purified from Methylocystis sp. strain SB2. Upon addition of copper, MB from ΔmmoD ΔmbnAN-mbnAS+pTJS-mbnPH bound one copper ion with a molecular mass of 913.14 (FIG. 11), consistent with earlier results. Loss of one proton following Cu²⁺ binding and reduction to Cu⁺ was also consistent with previous studies on the MB from Methylocystis sp. strain SB2 (Dershwitz, P.; Bandow, N. L.; Yang, J.; Semrau, J. D.; McEllistrem, M. T.; Heinze, R. A.; Fonseca, M.; Ledesma, J. C.; Jennett, J. R.; DiSpirito, A. M. Oxygen generation via water splitting by a novel biogenic metal ion-binding compound. Appl. Environ. Microbiol. 2021, 87 (14), e00286-00221)

Knock-In of Genes from Methylocystis sp. Strain SB2 into Chromosome of Methylosinus trichosporium OB3b

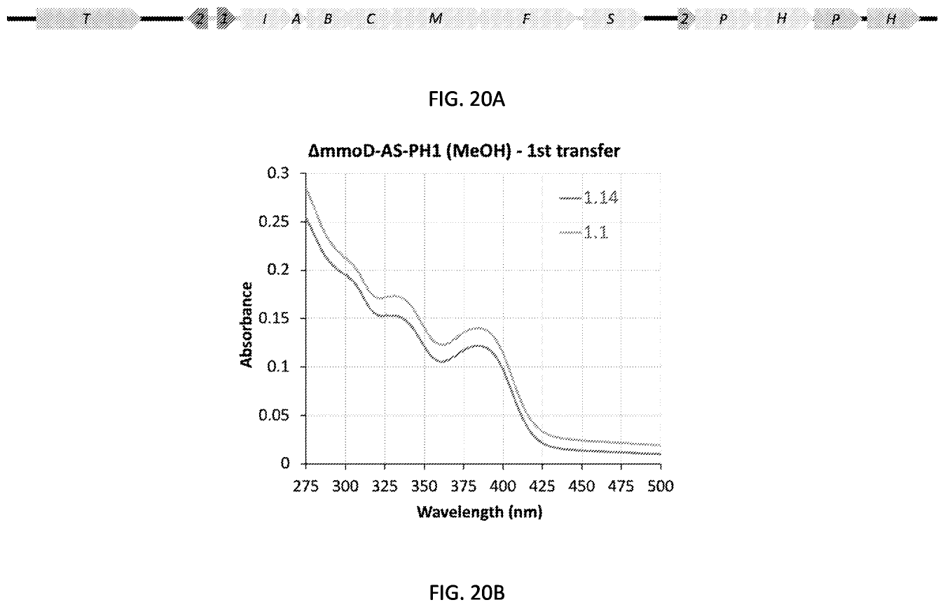

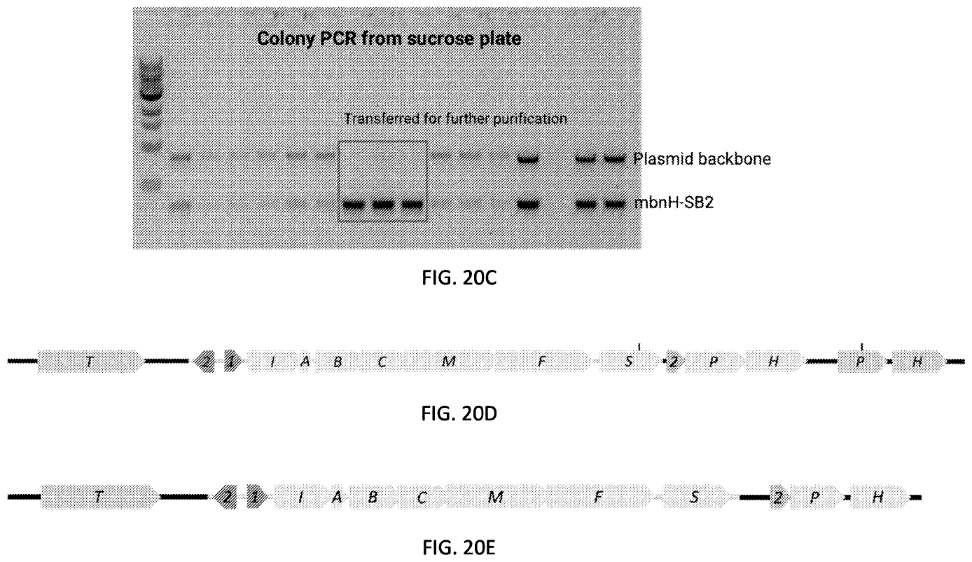

Genes from Methylocystis sp. strain SB2 (i.e., 2-1-I-A-B-C-M-F-S-2-P-H) shown in FIG. 20A were knocked into a mutant of Methylosinus trichosporium OB3b where native methanobactin (mbn) genes were removed (mbnABCMN) as well as mmoD. Three different constructs were generated where mbnPH were inserted in different locations relative to the OB3b backbone. In construct 1, shown in FIG. 20A, SB2 mbnPH were inserted next to the native OB3b mbnPH, likely causing a disruption of expression of native OB3b mbnPH. This construct is referred to as “ΔmmoD-AS-PH1”. As shown in FIG. 20B, clear evidence of methanobactin production from this strain is evident from UV-VIS absorption spectra. Note peaks at 336 and 386 nm, indicative of oxazolone and imidazolone rings of this form of methanobactin, respectively. FIG. 20C shows confirmation of successful deletion of native genes and replacement with heterologous genes from SB2. In construct 2, as shown in FIG. 20D SB2 mbnPH was inserted next to remainder of OB3b mbnN gene, hence no disruption of native OB3b mbnPH is thought to occur. In construct 3, as shown in FIG. 20E, native OB3b mbnPH was deleted. Constructs 2 and 3 showed similar efficacy for methanobactin production as construct 1, indicating that multiple strategies for where mbnPH are inserted are effective.

In sum, provided herein is alternative strategy for production of MB-SB2, including for use in treatment of Wilson Disease. Although methane is an inexpensive carbon source, its use for production of secondary metabolites (such as MB-SB2) is problematic given that yields of MB in wildtype methanotrophs are low, methane:air mixtures can be explosive, and overall microbial growth can be limited by mass transfer of gaseous substrates into solution. Herein, heterologous production of MB-SB2 is achieved with methane as the growth substrate in a mutant of M. trichosporium OB3b where native mbn genes and mmoD were deleted. Moreover, heterologous production is also achieved using methanol as the substrate. This finding is especially exciting as methanol, although more expensive than methane, can be more easily added, and indeed, MB-SB2 yields were greater on methanol than on methane in this mutant. Production of MB by trichosporium OB3b with methanol as the growth substrate was unexpected as previous efforts in our laboratories to produce native MB in M. trichosporium OB3b when grown on methanol have never been successful (data not shown).

In addition to identifying a key regulatory element controlling the production of MB, it is also shown for the first time herein that mbnPH is involved in the biosynthesis of MB-SB2. Such a finding contradicts earlier speculation that the products of these genes served to remove copper from MB after copper-MB complexes are re-internalized (Kenney, G. E.; Dassama, L. M.; Manesis, A. C.; Ross, M. O.; Chen, S.; Hoffman, B. M.; Rosenzweig, A. C. MbnH is a diheme MauG-like protein associated with microbial copper homeostasis. J. Biol. Chem. 2019, 294 (44), 16141-16151; Dassama, L. M.; Kenney, G. E.; Rosenzweig, A. C. Methanobactins: from genome to function. Metallomics 2017, 9 (1), 7-20). Without wishing to be bound by theory, MbnPH may be involved in oxazolone ring synthesis given that these genes are found in both Group I and II MB gene clusters, or it may have a role in removing the leader peptide from MB.

### Materials and Methods

### Growth Conditions

Wild type Methylosinus trichosporium OB3b and various mutants (Table 2) were grown in nitrate mineral salt (NMS) medium with or without 25 μg/ml kanamycin and in the absence (no added) or presence of copper (0.2 or 1 μM as CuCl₂). Methane and air were added at a methane-to-air ratio of 1:2. Cultures were incubated in the dark at 30° C. When methanol was used as the carbon source, methanol was added to NMS medium at a concentration of 0.25%. Liquid cultures were grown in 250-ml sidearm Erlenmeyer flasks with 20 ml NMS medium with shaking at 200 rpm. Growth was monitored by measuring the optical density at 600 nm (OD₆₀₀) with a Genesys 20 visible spectrophotometer (Spectronic Unicam, Waltham, MA). Triplicate biological cultures were prepared for all experimental conditions. Cultures were harvested at middle to late exponential phase for RNA isolation and transcriptional analysis of specific gene expression. Escherichia coli was grown in Luria-Bertani broth (LB) at 37° C. with or without supplement of 25 μg/ml kanamycin.

TABLE 2
M. trichosporium OB3b mutant strains used in this study.

| Strain | Description |
|---|---|
| ΔmbnAN | M. trichosporium OB3b with mbnAN deleted |
| ΔmmoD | M. trichosporium OB3b with mmoD deleted |
| | ΔmbnAN with mbnAS of Methylocystis sp. SB2 |
| ΔmbnAN-mbnAS | knocked in its genome |
| | ΔmmoD with mbnAS of Methylocystis sp. SB2 |
| | knocked in its genome and replaced its original |
| ΔmmoD ΔmbnAN-mbnAS | mbnAN |
| | ΔmbnAN-mbnAS carrying pTJS-140 with |
| ΔmbnAN-mbnAS + pTJS-mbnPH | mbnPH of Methylocystis sp. SB2 |
| | ΔmmoD ΔmbnAN-mbnAS carrying pTJS-140 |
| ΔmmoD ΔmbnAN-mbnAS + pTJS-mbnPH | with mbnPH of Methylocystis sp. SB2 |

mbn Gene Cluster of Methylocystis sp. Strain SB2 Analysis

Promoter and ribosome binding site (RBS) strengths were predicted using the Promoter Calculator (LaFleur, T. L.; Hossain, A.; Salis, H. M. Automated model-predictive design of synthetic promoters to control transcriptional profiles in bacteria. Nature communications 2022, 13 (1), 5159) and RBS Calculator, respectively (Reis, A. C.; Salis, H. M. An automated model test system for systematic development and improvement of gene expression models. ACS synthetic biology 2020, 9 (11), 3145-3156.)

Knock-In of Methylocystis sp. Strain SB2 Mbn Genes into M. trichosporium OB3b

mbnAS plus the two unknown genes were first knocked in a mutant of M. trichosporium OB3b where the native mbn genes—mbnABCMN—were removed using a sucrose counter-selection protocol (M. trichosporium OB3b ΔmbnAN). Briefly, the presumed methanobactin gene cluster—mbnABCMFS plus the two unknown genes (hereafter labeled mbnAS) of Methylocystis sp. strain SB2—and the upstream and downstream regions (arms) of mbnAN of M. trichosporium OB3b were PCR amplified using the primers listed in Table 2 (specific genes removed/added are shown in FIG. 14). The arms and mbnAS were digested with the appropriate restriction enzymes, ligated together and subsequently inserted into the pK18mobsacB mobilizable suicide vector (Schäfer, A.; Tauch, A.; Jäger, W.; Kalinowski, J.; Thierbach, G.; Pühler, A. Small mobilizable multi-purpose cloning vectors derived from the Escherichia coli plasmids pK18 and pK19: selection of defined deletions in the chromosome of Corynebacterium glutamicum. Gene 1994, 145 (1), 69-73). pK18mobsacB vector with mbnAS and arms was transferred to E. coli Top10 (Invitrogen, Carlsbad, CA). The plasmid was then extracted from transformed E. coli Top10 using the Plasmid Mini Kit (Qiagen, Hilden, Germany) following the manufacturer's instructions. The extracted plasmid was then transformed to E. coli S17-1.

Conjugation of E. coli S17-1 carrying the constructed vector with M. trichosporium OB3b ΔmbnAN was performed. Transconjugants were grown on NMS plates supplemented with 25 μg/ml kanamycin and 10 μg/ml nalidixic acid. The generated kanamycin resistant transconjugant colonies (after 10 days of incubation) was transferred to NMS plates with kanamycin and incubated for 7 days, and subsequently transferred to NMS plates with 2.5% sucrose (mass/vol). Sucrose-resistant colonies were generated after 10 days incubation and were screened for deletion of knock-in of mbnAS by colony PCR. Successful mbnAS knock-in mutants were further confirmed by PCR with DNA extracted from the mutant using the DNeasy PowerSoil Pro Kit (Qiagen, Hilden, Germany) following manufacturer's instructions.

In addition to knocking in mbnAS in M. trichosporium OB3b ΔmbnAN, a second production platform was used where mmoD, a gene in the mmo operon—encoding for the soluble methane monooxygenase—was also removed to create M. trichosporium OB3b ΔmmoD ΔmbnAN-mbnAS. Briefly, the E. coli S17-1 carrying pK18mobsacB with mbnAS and arms was conjugated with M. trichosporium OB3b ΔmmoD, described in Example 1 and in Peng, P.; Yang, J.; DiSpirito, A. A.; Semrau, J. D. MmoD regulates soluble methane monoxygenase and methanobactin production in Methylosinus trichosporium OB3b Appl. Environ. Microbiol. 2023. Transconjugants growing and selection of the marker exchange mutant (ΔmmoD ΔmbnAN-mbnAS) were performed as described above.

Heterologous Expression of mbnPH of Methylocystis sp. SB2 in ΔmbnAN-mbnAS and ΔmmoD ΔmbnAN-mbnAS

mbnPH from Methylocystis sp. strain SB2 was PCR amplified using the primers listed in Table 3. mbnPH was cloned into pTJS140 broad-host-range cloning vector (pre-digested with KpnI restriction enzyme) using NEBuilder® HiFi DNA Assembly Cloning Kit. The pTJS140 vector with mbnPH (pTJS-mbnPH) was transformed to E. coli S17-1 as described above. E. coli S17-1 with the constructed plasmid was conjugated with ΔmmoD ΔmbnAN-mbnAS. Transconjugants were grown on NMS plates supplemented with 25 μg/ml kanamycin and 10 μg/ml nalidixic acid. Selection and conformation of ΔmmoD ΔmbnAN-mbnAS with pTJS-mbnPH were done as described above.

TABLE 3
Primer used in this study.

| Primer | |
|---|---|
| name | Sequence (5′-3′)ª |
| mbnAS_F | ATTTTA ggtacc CATGATTCC |
| | TCTCCTGTCA |
| | (SEQ ID NO: 25) |
| mbnAS_R | ATTTTA tctaga GGAATGTCGC |
| | CTCTTAGCG |
| | (SEQ ID NO: 26) |
| mbnAN- | ATTTTT gaattc |
| armA_F | CGAAGGACAATAACAAGGCG |
| | (SEQ ID NO: 27) |
| mbnAN- | ATTTTT ggtacc |
| armA_R | ACTCCAAACAgcatgcGATA |
| | (SEQ ID NO: 28) |
| mbnAN- | ATTTTT tctaga |
| armB_F | ATCCTTCTATGTCTGCAGCC |
| | (SEQ ID NO: 29) |
| mbnAN- | ATTTTT aagctt |
| armB_R | GATCCTCCTCGAATTCCCTC |
| | (SEQ ID NO: 30) |
| mbnPH_F | agtgaattcgagctcggtac |
| | GGATTGCCGCCAACCATTG |
| | (SEQ ID NO: 31) |
| mbnPH_R | tctagaggatccccgggtac |
| | TTTCTTTGCTTTCCTGAT |
| | (SEQ ID NO: 32) |
| mbnA_F | AGCGCATCACCCTCAATGTC |
| | (SEQ ID NO: 33) |
| mbnA_R | ATGTCATAGGTGTCGCCGAG |
| | (SEQ ID NO: 34) |
| mbnB_F | AAGGCGTTCGATTGTCCAGT |
| | (SEQ ID NO: 35) |
| mbnB_R | GCGCAGAATATGGTCGGAGA |
| | (SEQ ID NO: 36) |
| mbnC_F | CGGACTGATCGCGACCTTTA |
| | (SEQ ID NO: 37) |
| mbnC_R | CGGAAAATCGACATGCCGTC |
| | (SEQ ID NO: 38) |
| mbnM_F | CGCAGAAATTCCAGCGAAAG |
| | (SEQ ID NO: 39) |
| mbnM_R | CAGCCGACTCCATAACCAAA |
| | (SEQ ID NO: 40) |
| mbnF_F | GAGTCCCGGTTCTGATCGTC |
| | (SEQ ID NO: 41) |
| mbnF_R | CAAACAACGCGTCCTCCAAG |
| | (SEQ ID NO: 42) |
| Unknown1_ | GCTCGTTCGGGTCATTCTTT |
| F | (SEQ ID NO: 43) |
| Unknown1_ | GCAACCTGATCATAGACGCATAA |
| R | (SEQ ID NO: 44) |
| Unknown2_ | GCTCGTTCGGGTCATTCTTT |
| F | (SEQ ID NO: 45) |
| Unknown2_ | GCAACCTGATCATAGACGCATAA |
| R | (SEQ ID NO: 46) |
| mbnS_F | AAGCGTCAATTCCGCTCCTA |
| | (SEQ ID NO: 47) |
| mbnS_R | GACGTGCGGATGCTGAAAAA |
| | (SEQ ID NO: 48) |
| mbnP_F | CGCGCATTCACTCGATTTAC |
| | (SEQ ID NO: 49) |
| mbnP_R | CCAGTCGTTCTGTTCCAGTT |
| | (SEQ ID NO: 50) |
| mbnH_F | GGACTTTGCCCGACTTCAT |
| | (SEQ ID NO: 51) |
| mbnH_R | CCATCTGCCTTTCCAGTGTTA |
| | (SEQ ID NO: 52) |
| pmoA_F | TTCTGGGGCTGGACCTAYTTC |
| | (SEQ ID NO: 53) |
| pmoA_R | CCGACAGCAGCAGGATGATG |
| | (SEQ ID NO: 54) |

RNA Isolation and cDNA Synthesis

RNA isolation was performed with a bead-beating procedure followed by column purification using an RNeasy Mini Kit (Qiagen, Hilden, Germany). Genomic DNA was removed from the column with RNase-free DNase (Qiagen, Hilden, Germany) treatment. Absence of genomic DNA was confirmed by 16S rRNA gene targeted PCR with extracted RNA samples as templates. Purified RNA was quantified using a NanoDrop 1000 Spectrophotometer (Thermo Scientific, Wilmington, DE). cDNA was synthesized from 200 ng total RNA using SuperScript™ III Reverse Transcriptase (Invitrogen, Carlsbad, CA) following manufacturer's instructions.

### Isolation of Methanobactins

Methylocystis strain SB2, and M. trichosporium OB3b were cultured and the methanobactins from each strained isolated. M. trichosporium ΔmmoD ΔmbnAN-mbnAS+pTJS-mbnPH was cultured in nitrate minerals salts medium with no amended copper and isolated as described above.

### Liquid Chromatography-Mass Spectroscopy (LC-MS)

Mass was determined in the negative ion mode on a Waters SYNAPT G2-Si High Definition Mass Spectrometer coupled to a Waters H-Class UHPLC system. Separation was achieved using a Restek Ultra C4 5 μm 50×1 mm column with a 5-100% mobile phase (0.1% formic acin/acetonitrile) gradient and a stationary phase of 0.1% formic acid in water.

### Other Methods

UV-visible absorption spectroscopy, copper titrations and acid hydrolysis were determined as previously described (Bandow, N.; Gilles, V. S.; Freesmeier, B.; Semrau, J. D.; Krentz, B.; Gallagher, W.; McEllistrem, M. T.; Hartsel, S. C.; Choi, D. W.; Hargrove, M. S. Spectral and copper binding properties of methanobactin from the facultative methanotroph Methylocystis strain SB2. J. Inorg. Biochem. 2012, 110, 72-82.)

### Abbreviations

MB: methanobactin
MB-SB2: methanobactin from Methylocystis sp. strain SB2
MB-OB3b: ethanobactin from Methylosinus trichosporium OB3b
ΔmmoD: markerless deletion of mmoD in M. trichosporium OB3b
ΔmbnAN: markerless removal of mbnABCMN in M. trichosporium OB3b
mbnAS: markerless insertion of mbnABCMFS of Methylocystis sp. strain S1B2 into the chromosome of Methylosinus trichosporium OB3b
pTJS-mbnPH: plasmid pTJS-140 with mbnPH of Methylocystis sp. strain SB2

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

We claim:

1. A mutant organism having attenuated expression of a native gene encoding methane monooxygenase component D (MMOD), wherein the mutant organism produces methanobactin when cultured in the presence of methanol.

2. The mutant organism of claim 1, wherein the mutant organism is a methylotroph.

3. The mutant organism of claim 2, wherein the mutant organism is a methylotrophic bacterium.

4. (canceled)

5. The mutant organism of claim 3, wherein the mutant organism is a member of the Methylocystaceae family, the Beijernickiaceae family, the Methylobacteriaceae family, or the Methylococcacea family of bacteria.

6. The mutant organism of claim 5, wherein the mutant organism is a member of the Methylocystaceae family of bacteria and belongs to a genus selected from Albibacter, Hansschlegelia, Methylocystis, Methylopila, Methylosinus, Pleomorphomonas, and Terasakiella, a member of the Methylococcaceae family of bacteria and belongs to a genus selected from Methylococcus, Methylomonas, Methylomicrobium, Methylotuvimicrobium, Methylocaldum, and Methylobacter, or a member of the Methylobacteriaceae family of bacteria and belongs to the genus Methylorubrum or Methylobacterium.

7. The mutant organism of claim 6, wherein the mutant organism is a strain of Methylosinus trichosporium, Methylococcus capsulatus (bath), or Methylorubrum extorquens.

8. The mutant organism of claim 1, wherein attenuated expression of a gene encoding MMOD comprises a deletion of mmoD.

9. The mutant organism of claim 1, wherein the mutant organism produces at least 50% more methanobactin compared to a wildtype form of the organism when cultured in the presence of methanol.

10. The mutant organism of claim 1, further having attenuated expression of one or more native genes in a methanobactin gene cluster, wherein the mutant organism expresses one or more heterologous genes in a methanobactin gene cluster from a different species of organism.

11. The mutant organism of claim 10, wherein the mutant organism has attenuated expression of three or more native genes in the methanobactin gene cluster and expresses three or more heterologous genes in the methanobactin gene cluster from the different species of organism.

12. (canceled)

13. The mutant organism of claim 10, wherein the mutant organism is a Methylosinus species or a Methylorubrum species and wherein the different species of organism is a Methylocystis species.

14. The mutant organism of claim 13, wherein the mutant organism is a strain of Methylosinus trichosporium or Methylorubrum extorquen.

15. (canceled)

16. The mutant organism of claim 10, wherein the one or more native genes are selected from mbnA, mbnB, mbnC, mbnM, mbnN, mbnP, and mbnH, and wherein the one or more heterologous genes are selected from mbnA, mbnB, mbnC, mbnM, mbnF, mbnS, mbnP, and mbnH.

17. The mutant organism of claim 16, having attenuated expression of native mbnA, mbnB, mbnC, mbnM, and mbnN.

18. (canceled)

19. (canceled)

20. (canceled)

21. The mutant organism of claim 10, wherein the mutant organism is a strain of Methylosinus trichosporium having attenuated expression of native mbnA, mbnB, mbnC, mbnM, and mbnN, and wherein the one or more heterologous genes comprise mbnA, mbnB, mbnC, mbnM, mbnF, mbnS, mbnP, and mbnH from Methylocystis sp. strain SB2.

22. (canceled)

23. (canceled)

24. (canceled)

25. (canceled)

26. (canceled)

27. A method of producing methanobactin, the method comprises culturing the mutant organism of claim 1 in the presence of methanol, such that methanobactin is produced.

28. (canceled)

29. (canceled)

30. A mutant organism having attenuated expression of one or more native genes in a methanobactin gene cluster, wherein the mutant organism expresses one or more heterologous genes in a methanobactin gene cluster from a different species of organism.

31. The method of claim 30, wherein the mutant organism is a Methylosinus species or a Methylorubrum species and wherein the different species of organism is a Methylocystis species.

32. The mutant organism of claim 30, wherein the one or more native genes are selected from mbnA, mbnB, mbnC, mbnM, mbnN, mbnP, mbnH, and combinations thereof and wherein the one or more heterologous genes are selected from mbnA, mbnB, mbnC, mbnM, mbnF, mbnS, mbnP, and mbnH.

33. A method of producing methanobactin, comprising culturing the mutant organism of claim 30 in the presence of methanol, such that methanobactin is produced.


SEQUENCE LISTING (54 sequences)

SEQ ID NO: 1 - protein, 125 aa, Methylosinus trichosporium
MRSGGRVARTTKKSGRTKMDQQTAQPEVRQTLIHADERYQAYTMDLEYMLRWEILRDGEFVQEGCSLSQESAREAVAHVLSHFRRQDATSQNDGGKSEAIRALLREIGTPEPLKDENGAAKPAHI

SEQ ID NO: 2 - DNA, 378 bp, Methylosinus trichosporium
  source 1..378: mol_type = genomic DNA
atgcgttcgggcggccgcgtcgcccgaacgacaaagaaaagcgggaggacgaaaatggaccaacagacggcgcagccggaagtccggcaaaccttgattcacgccgacgagcgctatcaagcctatacgatggacctcgaatatatgttgcgatgggagattctgcgcgacggcgagttcgtgcaggagggctgctctctgtcgcaggagtcggcgcgcgaggcggtcgctcatgtgctgagccactttcgccgacaggatgctacgtcgcagaacgacggcgggaaaagcgaagcgatccgcgcgctgctgcgcgagatcggaacgcccgagcccctgaaggacgagaacggcgcggcgaagccggcgcatatttag

SEQ ID NO: 3 - DNA, 90 bp, Methylosinus trichosporium
  source 1..90: mol_type = genomic DNA
atgactgtcaagattgctcagaagaaagtccttccggtcatcggtcgcgcggctgcgctttgcggttcgtgctatccgtgcagctgcatg

SEQ ID NO: 4 - DNA, 891 bp, Methylosinus trichosporium
  source 1..891: mol_type = genomic DNA
gtgctcgggggcggcactcgcctcttcggggcgattctggccgtattccacgaggcgaagatgcagatcggcttcaacttcacgctgaccggcacgctcgacatggtccagcagatgatcaaagagcggaaaatcgactatgtcgaaatgttgatcgacaatttcgttcatctcccgcccgagcagatcgcggattcattcgattgtcccgttgcatttcatatcatgctttcgaaatacctcgaaagagatcgggaggaattggagaagctcgggaagcggctgcgcagattcatcgacgtgatgcggcccgtctatgtctctgatcatattttatacttcacacacaacggcagaagccttttccatctcggcgaaatcgattattgagaatacgaccacgtgcggagcaaggtcgagcaatggcaggacatgctcggcacgcgcctctatcttgagaattatccgtcgatcatggatggcgcatgggacgcgccgtccttctacgagcggctgagccgggagaccggggtcggcgttctcttcgacgcgtcgaacgcgatctgcgctcagaacaacaccggcgcgccggtcgagctgtggaagaaaatcatcgagaccacgcgccatttccacgttgcaggatatggcacggctttcatcgagccgcgcgtcaaggccgacacgcacgaccgggagatggcggaggacacgctcgatttcctgagccggatgcgcacgtctttcgacaagcccggcgcgacgatcacttatgaacgggatttcgacattgactatgagtcgatctcggtcgacctgaaaagactgcgcgatattttcccctgcgtcgaggaagagcgtcatgagcctgttgcccactgcgccggttag

SEQ ID NO: 5 - DNA, 588 bp, Methylosinus trichosporium
  source 1..588: mol_type = genomic DNA
atgagcctgttgcccactgcgccggttagaatcgacgccgatctctacgatgatctcgcaaatcctgcccgccagtctctctatcccagggattcgcgcggcttcattcgcatcgatatcagccttcgggcctactggcacacattgttcgacacatgcccacgcctgctggagctctcggggccgagcggcggcgccatcttcctgcccttcatggcctgggcccgggagaacaacctcgctttcgactggagctttttcctctgggtctatgtctggctgcagcaatcggagttccgcgagcggctcgacgaggatcagctgctgcccgtcatgaccgcctccgcgacgcgatggttgatgatcgaccgcgacatcgatgcctgtcagatcgtgctgggaagccgctcgctcgccggcgcagccgtcgtcggcgccaagatcgacagcatccactgccggctcgagcaggtccagcaggtcgaattcgagaagccgctgccgctccccgacggcgagttcggctatttcctgacgcccggcttcgagatcgaccattttcccggctggcggccgctcccgcgatga

SEQ ID NO: 6 - DNA, 1311 bp, Methylosinus trichosporium
  source 1..1311: mol_type = genomic DNA
atgacgcggcccaaaacgggcttctggcgcggctactggccattgctcatggctctggcctcggctcaactcgcgcagcaagcggacatcgtgatgatgagccgcctcggcggccccgccgccggcgcttatgtgatgatgatgcgcctagcgctcgtcgaagtcgtgctgatgacggcgatgggcgcggtcgcatcgacggtggtcgcgcaggcgcaacgaaacggcggggccgcgccggtcatcggccgcatcctcggcctggccctgctgacgggactgctctgcggcgcgctaggctggctcctttatcccacggcggcgaggtggctggcgggggaaggcgacgtcgcgagcctcgtcggcgagggtgtgttctggcacgcgctggcggcgccctttcggtttgtggtcaacatcggcgcattcgtcctccacgccctcggtcgtggcccttcggtcgttcggtggaaattgaccgaggcggcggccaagatcgccgccaatctgctcctcatggatgttttgggatggggattttcgggctgcttcttgtccggcctcatcgtcggcgcgctgtcgtcgatctggtgccgccgcatgttgccgccgggcgacgcgaggctttcgcttcccggatgctcgtggacgctgcgcttccttcgtccgatctattgggaggcgcagcggctcatatccgtgcagctcgccatactcgcctgcttctccctgttcgcggcgccgtggctcgcgaattacagcgtctcgcggatgaattccttcgcggccggccagaccttgatgctgatgctgttcacgcccttcgtcgcgctgacgcgattcctggcgttccggcacgctgcgctgcgcgacggccggctctcggcgtcgatgtggagagtgtgggcgcagggcgcgcctgtcgtggtcgccgccgccctcgccttgctggagagcgacgagctgctcggtcgcttatatggccagagcgggcattggtggtcgacgctgatccaggcgctggcgatttcgcttccccttcgcttcgcggcgaatatcgcccgagccttgttccattcgcagggctcgttcgccatggtcgcagccgccgacggcgccgcgttttggctgatcgcgacgccgcttgtcgccatcgggctctacgtcgactccccggccgtcgcttattcctcgctgatccttccagaagcggcctgcgccgcttggatgtggggccggctcggaattctcgctttccgtgaaaaaggaccggtcggcgccgacgaaggcgtgaaagctttgctcgaatga

SEQ ID NO: 7 - DNA, 1125 bp, Methylosinus trichosporium
  source 1..1125: mol_type = genomic DNA
atgacggcgatcccatgtgaaacgaacgggctcacgagcgagccgatcgcgatgcgagccaaatcgagcctggccgtcccgatattcgccgttgcgccgcggcccgacggcatcttgctcgacagcaacgaatttcgctttcctctcccgaagaggtttctgaaacgcgtcgcggacgagctgtggccggtcgatctgcgatgctatcctgatttcgccgaagtggaaaaaatcaaggtcgccttggcgcgagacctcggcgtcgagcccgacaatctgttgctcggggtcggctcgaccgaactcctcgatttgatcagccgcgctttcggcgaaagggaccacagaatcgtcacgccgattccatcctttccgatgtatgctcattacgccaatctgaacgatcgaaaattcgttcccgttccgctcgacgacgatctctccttgtcttcgaaagtggcggaggcgctgctgcgggagcccgggagcaaatccttctatgtctgcagcccgaataatcccaccgggctttccatagagcgagagacgttcgacgctctcgcttccgatcctcgcgccattttgtgcctggacgaggcatacgccgaatattccggccgctccttcgtcacggaggcggcgacacgcgacaatgtcgtcgtcacgcgctccttttcgaagatcggcctcgcgggatgccggttcggctatgcggtcgcctcgaaaaatctcatccgggaactcgcgaaggccgctctgccctatgctctgaactccctcaccttgcgcattgcgcaaatcttcatcgaggagatgccgctgatcagagcggtcgtcgaggacgtcaagcgtgaacgggcgcgtctttcgaccgcgctggcgaacctgcccggtttccacccctaccgatccgacgccaacttcctgctctgcgcggcgcctggcgacgccgccgcgctcgcccgaaggttgatcgagctacgaaaaatctgggtgcgacccttttcgccccaatcgcgcttacgcgactgtctgcgcatcgcggtcggttcgccggaggacaatgacatccttctagacgccttgagacaggaatcgacgatccacgcgcctcggtcggaatga

SEQ ID NO: 8 - DNA, 975 bp, Methylosinus trichosporium
  source 1..975: mol_type = genomic DNA
atgtgggaattgcatagagcgcggaccgcgacaattgccgccggagcgctgattgtcgccggcgcggcgctggccgcgggcgtgaagacacagccggtcgcagtccgtttcgcgctcgtcgccgatggcaaggaagtgggctgcggcgcgccgctcgcaaacctcggcagcgggcgcctcgccggcaagctgcacgaagcgcgcctctacgtctatggcttcgagctggtcgacgccaaagggaagcacacacccatcgcactgacgcagaacgattggcaatacgccgatgtcgcgctgctcgacttcaaggacgcgcgcggaggcaacgccgcttgcacgccgggcaatccggccaagaacacgaccgtcgtcggagccgcgccgcaaggcgcctatgtcggtctcgccttctctgtcggcgccccggtggagagcctcgtcgacggcaagcccgtcttcgtcaaccattccaatgtcgaggccgcgccgccgcctctcgacatttccggaatggcgtggaactggcaggccggccgccggttcgtgacgatcgaggtcattccgccggcggcggtgatcaagcccgacggctccaagtcgcggacctggatggtccatgtcggctcgaccggctgcaagggcaatccggcgaccggcgagatcgtcgcctgtgcgcatgagaaccgctttcctgtcgtcttcgaccgcttcgatccgaagacgcagcgcgtcgagctcgatttgacgacgctgttcgagagcagcgacatcagcgtcgacaagggcggcgcggtcggctgcatgagcgcgctcgatgatcccgactgcccggcggtgttccgcgcgctcggcctcaatctcgccgacagcgcgcccggcgccaatgacgccggcaagccatcgaggcccggcgtctcgccgatattctctgtcggcgccgcagcgtcgaaagtcgcgggcggcaagcaatga

SEQ ID NO: 9 - DNA, 1143 bp, Methylosinus trichosporium
  source 1..1143: mol_type = genomic DNA
atgatgcgcgcgtcgtttctcgtactcgccgcgctcgttctcggcgcgcgctctgcggccgagccggcccccgcctggaattgggacttaccgaaatacatcccgccgccgcgtgttcccgtcgacaatccgatgtcggaggagaagttccagctcgggcggcgcctcttctacgataagcgcctctccggcaatggaacgctctcgtgcagctcctgccatctgcaggagcgcgcctttaccgacggacgcaccgtctccatcggctcgacgggcgcgaagacgccgcgcaatgcgccgtcgatcgcctattccggctggcatggaacgctcacctgggccaatccggcgctggtgacattggagcggcagatgctcaatccgctgttcggcgccgatccgatcgagatgggcgcgagcgacgccaataaggcggagatcgtcgcgcgctttcgcgccgacgccgattatcgccgatggttcgccgcggcctttcccgaaatgagcgagcccatctccttcgcgacgatcatcgcggcgatctcggcgttccagcgcggcgtctattccttcgacagccgctatgatcattatctgcagggcgaggcgcagctcaccgaagccgagcagcgcggacatgatctttatttcggtgagaaggcggaatgtcatcactgccatggcagcgtcggcctcgacgatcaattcgtgcatgcgaggacgcgcgagccggagctgccgttccataacaccgggctctacgacattgacggaaagggcgcttatcccgcgcccaatcacgggctcttcgacatcaccggcgatccggacgacatgggcaagttccgcgcgccgagcctgcgcaacatcgcgctgaccgcgccttacatgcatgacggcagcgtggcgacgctggaagaggtgatcgacatttattccgaaggcgggcgtaagatcgcgagcgggccgcatgcgggcgacggccgcgccagtgcgctgaagagcgggctgatcgtgaagatcgatctgacggcgcaggagaaggctgatctcctcgccttcctgaagacgctgaccgacgagagcctgatcgcctcgccgcgtttctctgatccctggagaacgccgaccgctgcgaaatga

SEQ ID NO: 10 - DNA, 93 bp, Methylocystis sp.
  source 1..93: mol_type = genomic DNA
atgaccatccgtatcgccaagcgcatcaccctcaatgtcattggccgcgccagcgcccgctgcgcgtcgacctgcgccgccaccaacggctaa

SEQ ID NO: 11 - DNA, 825 bp, Methylocystis sp.
  source 1..825: mol_type = genomic DNA
cgacttggcttcaatttcacgctcggcgacacctatgacatggctcagaggctgatcgccgaagggcacatcgattattgcgaatttcttatcgacaattttttctgcgtcgatccggacgaattggccaaggcgttcgattgtccagtcgggttgcacattatgtattcgaagtttctcgaagccgatcggccgacgctcgtcgatttcgccgctcggcttcgagattacatcgacgcgctgaacccgatttatgtctccgaccatattctgcgcttttctcacgaggggcgcgccttctttcacttaggcgaaatcgattattccgccgaatatgaatccgtgcgcgacaaggtcgtgcaatggcaggaaatcgtcggacagcgcattcattttgagaattacccgtccatcatggacggcgggttggaagcgcccgcttttttcgaacgcctcatgaaggaaacaggcgcgggcgtgctgttcgacgcctccaacgccgtctgcgccaatcgcaattgcgggacgccgttcgaggcctggcgcaacgtgatcgcgaaagcccagcatttccacgtcgccggctatcggcagtccttgatcgagcccttcatttcgctcgacacccatgccgaggcgttggcgccggacacgctcgccttcctccagaattttcgatcggtcttcgacaagccgggcgcaacgatgacttatgagcgcgacgatcaaatcgagttcgacgatatcgtcgtcgatctgaaggcgttgcgcgatctgttcggccaaccagaagaaacgcgtcatgaccttgctctcactgcctag

SEQ ID NO: 12 - DNA, 582 bp, Methylocystis sp.
  source 1..582: mol_type = genomic DNA
atgaccttgctctcactgcctagtcggactgatcgcgacctttaccctgcgctgatgtcgccgcagctgaacgcaaaatatcctgcggaccacaaggcgttcgtgcggatcgacgtgagcctgcgcatctattggcacacgctcttcgacatttgcccagaactgctggagctttccggtccggacggcatgtcgattttccggcctttcatggcctgggctgaagagaagaagctcgccttcgactggtcctattatctttgggtctatgtttggctgcgccaatcgccgttcaaggaacggctgttggcggacgacaaatatctccgatcgatcatggcggcctccgccgcgcggtgggcgatcctcgatcgcggtcgaagccgcgggatcgtgatcggatgcgccgatacggcggatctcgttatcggctggaaatgccgcaacgttcgcctcggacgagagatcgagcttatcgaacccgaggagccgttgccggcgccgccagacctcttcggctactttacgctgccgagctttgaactcgacatatttccggggtggcagagcctcgcgcgatga

SEQ ID NO: 13 - DNA, 1302 bp, Methylocystis sp.
  source 1..1302: mol_type = genomic DNA
atgacaagcgcgccgctgtggcgcggatactggttcctgttcgccagcacgtccgcagccgccttcatcatgcagatcgatctcgccatggtcgcgagattgggcggccgcgcgtcaggcgcctatgcgattctgatgcgcgtcacgctgctcgatgtcgcggtcaccatcgcttccgcggccgtcgccgccatcgcgctcggccatgcgcaaaagaagggcgagacggccgacgcgatcgaaaagacctgcgcgctctccctcgcgcttggcgtcgcgacagcgattttcggcttcttcgcctatccgatcttccttgacgccctgatgggcgacgccgccgccgctccctatatcggcgccccgatcctttggctcgtcgccggcgcgccgttccgcgttctatcgaacacgcaaggattcctgttgcacgcgcttggacgcggcggctcggttctcacatggaagctcgcagaaattccagcgaaagccggcgccaacgtcctgttcatgcaaaccttcggattgggttttaccggctgttttctcgcgggctgcgtcgtcgccctcgcctcgtctttttggttatggagtcggctgcacgcgcatggcgcgcgcacattgcgccttcccgaactcgccttcgcgcgctcctttctgagcggaaccttctgggaagcgctgcgggtgctgtcgccgcaggcggcgatgctcttctcgctgacgctcttttcgctgccctggcattacgccgcgaacggccaacgcctcgactcctacgcggccgcgcagaccttcatgctgttcattcttggtccgctcattacgctgatgcgctatctcgccatttaccttcccaagcaatctcccgaggattggacgccagcccttgcgccggtgatgcgcgtcggcgcgccactcgcgctgacggccgccgtcttgctgctatcggggcgcgattggatcggggacgctctctaccgccaacacggaccctggtggtcggttttcgtcgcctgcttggcgatttccatacctatccgattcatcggcagcatcgtccgaggactaacgctcgctcggagcgccttcgcagaactgacctccgtcgatgtgcttgcgcagtggctcatcgcgccgctgttcattctgttcggcctctccgtaaaccgtccggagatcgcctatcaatcgctgatctggcctgaaatcgtcgctgttttcttgctctggcgcggacttcgatccgcgcccgaagagcccgtcggcgtcccgtggcccgctaaaggacatgtgcaatga

SEQ ID NO: 14 - DNA, 1647 bp, Methylocystis sp.
  source 1..1647: mol_type = genomic DNA
atgagcgtggagcgagtcccggttctgatcgtcggcgccggctatgccggtctgtctgcggcgacgttgcttgcatggcgcggcgtcccctgccgtctcgtcgagcgacgcgcgtcaacgtcgcgactgccgaaagcgcatgggatcaaccgccgcagcatggaagttctgcgcgtcgtccctggcttggaggacgcgttgtttgcggcaagtcgcgccggcgccaacgaatccacgctcatcatcgccgagtcggtcacgagcccgccgatcgaaacgctcgtcaccaagatctcgctcgacgcaacgccggtgtctcccagcagaatatgcaccgcaggccaggatcgagtcgaacccgtgctgctgcgcttcgcccgcgagaacggcgccgacgttcgattttcgacgacgctagagcgcttctcgcagcgggacgacggcgtcgacgcgatcctgcgcgatgaagcgtcgggacaagagaccaccgttctcgcggactacatgatcgccgccgacggcgccggcggaacgatccgcgatgtcggcggcgtcaagatggaaggtcccggcgttctcgccgatacgatttctgtgctgttcgaggccgatctcgactcgatcctgccaggcgggggatttgcgctctattatctgcgcaacccggcgttcagcggcgccttcgtcacctgcgacgaacccaatcacggccagatcaacatcgaatatgattccacgcgcgatcaggcgtccgatttcgacgaagaacgatgcgaagcgctcgtgcgacaatctcttggcgtcgccgatcttgccgtaaagattctcgatattcgcccctggcagatggccgctctcctcgccgatcgcatgtctttcgggcgggtgtttctcgcgggcgactgcgcgcacatcacgccgcccgttggcgggctcggcggacaaaccgcgatccaggacgccgccgacctggcgtggaaacttgcgctcgtcgtcaagggccaggctgcgcccacgctgctcgacagctatgagatcgagcgcaggcccgtggcgcggatcgcaatcgcgcggtccatcgccaattacgtcgagcgcctcctgcccgatcgtcaggacatccgcattcgggaagacgaatacggcctgctcgaaactgctatgggctatcgctaccgctccgacgcgatcatcgctgatgagttcgacgacggcgcatgcgtcgaagatcctcttcgcccgagcggcgcgccgggaacgcgtctggcgcatgtctggttgcggcgcggcgaggagacgatctcctctcacgatctgatcggccgcgatttcatgctttttaccggccccgatggcggcgactggatcgaggcggcgcggcgcatcgccctgcgctcgaaagcgccgcttggcgtctgccgcctcggcttcgacgtcgatgatcccgaagggctgtttctgccgcgcctgcgcatttcgcctgaaggcgcgctcctcgtgcgtcccgacggctacatcgcctggcgttcgcgaggccgaagtcccgatccgttcgcaacgctcgaagcgagttttgcgcgcgtgcgcggtttcgacacgggccaatccagcggcagtcacgcggctgcgttcgccgacgcaagctag

SEQ ID NO: 15 - DNA, 1011 bp, Methylocystis sp.
  source 1..1011: mol_type = genomic DNA
atgtccatgctgtcgccggagctccacgcgcctttccggtcggaaccgatcctggcggttgatccgaagcggctcgaaacgatccaggccccctaccaggacttcaaccgcctgtcgcgggccagcggcgctttggagaccctgccccttcgagcgaagcgtcaattccgctcctattattttttcaatccgccaaaatggcgcgtctgggcgcgttcgctcggctccggcgaacgcatggcgccgtccttcgcatcgatcggcgcggtgcgctccggcacgtctttcctctcctcctatatttttcagcatccgcacgtcgtgctgccgctgtcgaaggaaatctccttcacggagacgatgcgcgaactcatggcgcattttccgacgcttgcggcgcaaagggcggcggagcgacgcaacggcggcgcgatcaccggctactgcacgccggtgatgcccaatccactttggatatttctggcgcagtcgatgtttccagacatgcggatcatctgcgttctgcgcgatccggtcgagcgcactttttcgcactggcgatgggatcaaaagcgcttcatcagccgcaagtcgcaggacccgcattggaagggctttccggaccttcaaaccttgatcgaagccgaaaaggcgataatacgcggcggcggcatggagccgcacgcgttttccggcgcgcgggccggctatctgcgccacagcatctacgcgccctttctgcgcctgctgttcgagaaattcggcagagactgcgttttcatcgtcgatgcggcggaattttttcgcgacccgcgatcgaccgcgaaggagatttacgcgtttctcggccttcccaacgtcgagccgctggtgatcgaggagcaaaatccgggtccccccggaacgcttgaggacggacttcgggagaatttggccgagttcttccggccctacaatgaagagctctatgctctgctgggtagggatttcggctggggcgccggccgctaa

SEQ ID NO: 16 - DNA, 1056 bp, Methylocystis sp.
  source 1..1056: mol_type = genomic DNA
atgcgcgcattcactcgatttaccgccgttatcgccggtctcgcggccattggcggcgcgcaggcggcggcgaagggcgacggcggcgaaaggcagcaggtcgcgatacgctttgcgcttaccgcgggcgatcagccggtcgaatgtggccgcgacattgccgggctaggaaccggcgggcagccggcgcagctccacgacgcgaggttctacatcgccgcgccggcgctgatcgacgccgccggccacgaagtccccatcgaactggaacagaacgactggcaatacgccaacctcgctctgctcgatttcgaaaacaagaccggcaaatgcgtcggcagcgccgacatcaacgacacgatcaagggatcggcgccgcgcggccgttacaaagggttcagcttcgtcatcggcgtcccgagcgttgtccaggacaaggacggcaaggacgtcattctcaaccactcgaatttcgccaccgctcccgcgccgctcgatcttcagtcgatgacgtggaactggcaggccggccgcaaattcataaagattgaagttgatccggatggcggcgtgacccgaccgccgcccccagcgaaaaaccccgccatcgccaccagtggcgcaggcgaaaaaactgacggcccgccgccgatggcggacgcgccgaagacggcgccgctgcgcgtcaactcggacgggacgatcaccgtctcgacctggatgctgcaccttggttcgacgggctgtcgcggcgacgcgacgaaaggcgaaatcacctcctgcgccaatccaaatcgctttcccgtgaaactcgcgtccttcgaccccgcgcggcagcgcgtcgtgctcgatttgaagccgctgttcgccggcatcgatcttggcaaagaccagggattctcgaccggttgcatgagtggtccggccgatccggaatgcacgccgatgttcgaaaatctcgggctacggctcaaggaaaccgcgcccgacgccaatgacgccggccagtcctccggcgcgtcgacgaagattttccgcgcggaggtcacgaaatga

SEQ ID NO: 17 - DNA, 1179 bp, Methylocystis sp.
  source 1..1179: mol_type = genomic DNA
atgaagcgcgaagctctcattctcctcttcgccgcaacgctgatcgcaggcggggcgcaagccgaccctgtccccagcgcggagtcctggcgctggactttgcccgacttcatgccgccgccacgggttccagccgacaacgcaatgtcggaggcgcgttttcagctcggacgcaaactcttctacgacccgcgtctgtccggaaacggcaaggagagctgcgcctcctgccatgagcaacgtctggcgtttactgatggccgtccggtcgccgtcggcgccacgggccagcatacgccgcgcaattcgcccagcatcgccaacgccgcttggcgcgccacgctcacctgggcgaatccggcgatggtaacactggaaaggcagatggaaggcccgctctttggcgaagagccgatcgagatgggcgtcaatgacaagaaccgcaaagagatactcgcgcgtttcgacagtgacgcagagtatcgccgcgacttcgcggccgcctttcccggcgaggcggactcgatcagcttcggcaacgtcatcaaggcgatctccgctttcgagcgcggcgtcgtctcggcgtcctcgaaatatgatctctatcttgaaggcaaggcgacgctctctccagaagagacgcgcggcaaggatcttttcttcggcgagaaggccgagtgccaccactgccacggcagcgtgaatttcgacgatcagttctatcacgccaaaacgcgcgagatcgagacgccgtttcacaacaccggcctctatgatctcgatggcaaaggcgcctatcccgagcccaatcgcggcgttttcgagaacacgcataatcccgccgagatgggcgcgtttcgcgcgccgagcctgcgcaatatagctgtcaccgcgccttacatgcacgacggcagcgtcgcgacgctcgatgacgtgatcgacatttacgcggccggcggacgcaatgtcacgagcggtccactcaagggcgacggtcgctacaacgcaaacaaaagcgcgttgatcgttcccatcgatctgtcgcctcaggaaaagagcgatctgatcgcctttctgaaaacgctgaccgacgagtcgttgctgacatctccacgtttcgccgacccctggaaaccggcgtcgacggcaagcgggccatcgcgtgcgtcctcccattga

SEQ ID NO: 18 - DNA, 12 bp, Synthetic construct
  source 1..12: mol_type = other DNA
atttttgaattc

SEQ ID NO: 19 - DNA, 21 bp, Synthetic construct
  source 1..21: mol_type = other DNA
ggcaccaaattcaccatcacc

SEQ ID NO: 20 - DNA, 12 bp, Synthetic construct
  source 1..12: mol_type = other DNA
atttttggtacc

SEQ ID NO: 21 - DNA, 20 bp, Synthetic construct
  source 1..20: mol_type = other DNA
gaacgcatgtctttcgcaat

SEQ ID NO: 22 - DNA, 30 bp, Synthetic construct
  source 1..30: mol_type = other DNA
atttttggtacctgctgcgcgagatcggaa

SEQ ID NO: 23 - DNA, 12 bp, Synthetic construct
  source 1..12: mol_type = other DNA
atttttaagctt

SEQ ID NO: 24 - DNA, 22 bp, Synthetic construct
  source 1..22: mol_type = other DNA
gacggatgaagaattcgagctg

SEQ ID NO: 25 - DNA, 31 bp, Synthetic construct
  source 1..31: mol_type = other DNA
attttaggtacccatgattcctctcctgtca

SEQ ID NO: 26 - DNA, 31 bp, Synthetic construct
  source 1..31: mol_type = other DNA
attttatctagaggaatgtcgcctcttagcg

SEQ ID NO: 27 - DNA, 32 bp, Synthetic construct
  source 1..32: mol_type = other DNA
atttttgaattccgaaggacaataacaaggcg

SEQ ID NO: 28 - DNA, 32 bp, Synthetic construct
  source 1..32: mol_type = other DNA
atttttggtaccactccaaacagcatgcgata

SEQ ID NO: 29 - DNA, 32 bp, Synthetic construct
  source 1..32: mol_type = other DNA
attttttctagaatccttctatgtctgcagcc

SEQ ID NO: 30 - DNA, 32 bp, Synthetic construct
  source 1..32: mol_type = other DNA
atttttaagcttgatcctcctcgaattccctc

SEQ ID NO: 31 - DNA, 39 bp, Synthetic construct
  source 1..39: mol_type = other DNA
agtgaattcgagctcggtacggattgccgccaaccattg

SEQ ID NO: 32 - DNA, 38 bp, Synthetic construct
  source 1..38: mol_type = other DNA
tctagaggatccccgggtactttctttgctttcctgat

SEQ ID NO: 33 - DNA, 20 bp, Synthetic construct
  source 1..20: mol_type = other DNA
agcgcatcaccctcaatgtc

SEQ ID NO: 34 - DNA, 20 bp, Synthetic construct
  source 1..20: mol_type = other DNA
atgtcataggtgtcgccgag

SEQ ID NO: 35 - DNA, 20 bp, Synthetic construct
  source 1..20: mol_type = other DNA
aaggcgttcgattgtccagt

SEQ ID NO: 36 - DNA, 20 bp, Synthetic construct
  source 1..20: mol_type = other DNA
gcgcagaatatggtcggaga

SEQ ID NO: 37 - DNA, 20 bp, Synthetic construct
  source 1..20: mol_type = other DNA
cggactgatcgcgaccttta

SEQ ID NO: 38 - DNA, 20 bp, Synthetic construct
  source 1..20: mol_type = other DNA
cggaaaatcgacatgccgtc

SEQ ID NO: 39 - DNA, 20 bp, Synthetic construct
  source 1..20: mol_type = other DNA
cgcagaaattccagcgaaag

SEQ ID NO: 40 - DNA, 20 bp, Synthetic construct
  source 1..20: mol_type = other DNA
cagccgactccataaccaaa

SEQ ID NO: 41 - DNA, 20 bp, Synthetic construct
  source 1..20: mol_type = other DNA
gagtcccggttctgatcgtc

SEQ ID NO: 42 - DNA, 20 bp, Synthetic construct
  source 1..20: mol_type = other DNA
caaacaacgcgtcctccaag

SEQ ID NO: 43 - DNA, 20 bp, Synthetic construct
  source 1..20: mol_type = other DNA
gctcgttcgggtcattcttt

SEQ ID NO: 44 - DNA, 23 bp, Synthetic construct
  source 1..23: mol_type = other DNA
gcaacctgatcatagacgcataa

SEQ ID NO: 45 - DNA, 20 bp, Synthetic construct
  source 1..20: mol_type = other DNA
gctcgttcgggtcattcttt

SEQ ID NO: 46 - DNA, 23 bp, Synthetic construct
  source 1..23: mol_type = other DNA
gcaacctgatcatagacgcataa

SEQ ID NO: 47 - DNA, 20 bp, Synthetic construct
  source 1..20: mol_type = other DNA
aagcgtcaattccgctccta

SEQ ID NO: 48 - DNA, 20 bp, Synthetic construct
  source 1..20: mol_type = other DNA
gacgtgcggatgctgaaaaa

SEQ ID NO: 49 - DNA, 20 bp, Synthetic construct
  source 1..20: mol_type = other DNA
cgcgcattcactcgatttac

SEQ ID NO: 50 - DNA, 20 bp, Synthetic construct
  source 1..20: mol_type = other DNA
ccagtcgttctgttccagtt

SEQ ID NO: 51 - DNA, 19 bp, Synthetic construct
  source 1..19: mol_type = other DNA
ggactttgcccgacttcat

SEQ ID NO: 52 - DNA, 21 bp, Synthetic construct
  source 1..21: mol_type = other DNA
ccatctgcctttccagtgtta

SEQ ID NO: 53 - DNA, 21 bp, Synthetic construct
  source 1..21: mol_type = other DNA
ttctggggctggacctayttc

SEQ ID NO: 54 - DNA, 20 bp, Synthetic construct
  source 1..20: mol_type = other DNA
ccgacagcagcaggatgatg